(19) United States
(12) Patent Application Publication
(10) Pub. No.: US 2023/0250147 A1
(43) Pub. Date: Aug. 10, 2023

(54) GLP-1R AGONISTIC PEPTIDES WITH REDUCED ACTIVITY

(71) Applicant: SANOFI, Paris (FR)

(72) Inventors: Oliver BOSCHEINEN, Frankfurt am Main (DE); Werner DITTRICH, Frankfurt am Main (DE); Andreas EVERS, Frankfurt am Main (DE); Thomas LANGER, Frankfurt am Main (DE); Christine RUDOLPH, Frankfurt am Main (DE); Uwe SCHWAHN, Frankfurt am Main (DE); Mark SOMMERFELD, Frankfurt am Main (DE)

(21) Appl. No.: 18/013,479

(22) PCT Filed: Jul. 2, 2020
(86) PCT No.: PCT/EP2020/068747

§ 371 Date: Dec. 28, 2022

(51) Int. Cl. C07K 14/605; A61P 1/16; A61P 3/10

(52) U.S. Cl. CPC C07K 14/605; A61P 1/16; A61P 3/10; A61K 38/00

(57) ABSTRACT

The present invention relates to Glucagon-Like Peptide-1 Receptor (GLP-1R) agonistic peptides with reduced GLP-1R CA agonistic activity, and fusion molecules comprising the same. The present invention also relates to nucleic acid molecules encoding GLP-1R agonistic peptides with reduced GLP-1R agonistic activity, pharmaceutical compositions and combinations comprising GLP-1R agonistic peptides with reduced GLP-1R agonistic activity, and kits including GLP-1R agonistic peptides with reduced GLP-1R agonistic activity. The present invention further relates to the use of GLP-1R agonistic peptides with reduced GLP-1R agonistic activity as medicaments, in particular, for the treatment of obesity, being overweight, metabolic syndrome, diabetes mellitus, diabetic retinopa-CA thy, hyperglycemia, dyslipidemia, NASH and/or atherosclerosis.

### TECHNICAL FIELD OF THE INVENTION

The present invention relates to GLP-1R (Glucagon-Like Peptide-1 Receptor) agonistic peptides with reduced GLP-1R agonistic activity, combinations and fusion molecules comprising the same, as well as to corresponding nucleic acid molecules, pharmaceutical compositions and kits. It further relates to the use of GLP-1R agonistic peptides as medicaments, in particular for the treatment of obesity, being overweight, metabolic syndrome, diabetes mellitus, diabetic retinopathy, hyperglycemia, dyslipidemia, Non-Alcoholic SteatoHepatitis (NASH) and/or atherosclerosis.

### BACKGROUND OF THE INVENTION

The use of GLP-1R agonistic peptides alone or in combination with other active pharmaceutical ingredients can have drawbacks. GLP-1R agonistic peptides are already pharmaceutically effective at lower plasma levels. At higher plasma levels, GLP-1 (the primary GLP-1R agonist) is known to have adverse effects, e.g., it induces nausea and vomiting. In contrast, the pharmacological effects of other active pharmaceutical ingredients that may be combined with GLP-1R agonistic peptides, e.g., of Fibroblast Growth Factor 21 (FGF21) compounds, are often observed at higher plasma levels than the plasma levels of GLP-1 that exert pharmacological effects. Taken together, this indicates a risk of GLP-1-mediated adverse effects when administering a GLP-1R agonistic peptide alone or in combination with another active pharmaceutical ingredient, e.g., an FGF21 compound and a GLP-1R agonistic peptide in the form of a fusion molecule. Accordingly, new GLP-1R agonistic peptides that overcome these challenges are needed.

### SUMMARY OF THE INVENTION

It is an object of the present invention to provide GLP-1R agonistic peptides with reduced GLP-1R agonistic activity. Such GLP-1R agonistic peptides can, for example, be used for balancing the GLP-1R agonist/FGF21 compound activity ratio in order to achieve the beneficial effects of both active agents (e.g., in terms of body weight, lipids, and/or glycemic control and the like) while avoiding potential adverse effects (e.g., nausea and/or vomiting and the like).

In one aspect, the present invention relates to a GLP-1R agonistic peptide having a GLP-1R agonistic activity which is about 9- to about 531-fold reduced as compared to the GLP-1R agonistic activity of a native GLP-1(7-36) (SEQ ID NO: 260), wherein the GLP-1R agonistic peptide comprises or consists of the amino acid sequence

(SEQ ID NO: 635)
X₁-G-E-G-T-F-T-S-D-X₁₀-S-X₁₂-X₁₃-L-X₁5-X₁₆-X₁₇-X₁₈-X₁₉-
X₂₀-X₂₁-F-X₂₃-E-W-L-X₂₇-X₂₈-X₂₉-G,

wherein

### X₁ is H, Y or F,

### X₁₀ is K or L,

### X₁₂ is K, I or Q,

### X₁₃ is Q or L,

### X₁₅ is E, A or D,

### X₁₆ is E, K or S,

### X₁₇ is E, R or Q,

### X₁₈ is L, A or R,

### X₁₉ is V, A or F,

### X₂₀ is R, H, Q, K or I,

### X₂₁ is L, E, H or R,

### X₂₃ is I, Y or F,

### X₂₇ is I, L, K or E,

### X₂₈ is A, K, N or E, and

### X₂₉ is G, T, K or V;

wherein, optionally, the amino acid sequence further comprises at least one additional amino acid residue at its N-terminus; and
wherein, optionally, the amino acid sequence further comprises a peptide extension consisting of up to about 12, about 11 or about 10 amino acid residues at its C-terminus.

In one embodiment, the GLP-1R agonistic peptide comprises or consists of the amino acid sequence

(SEQ ID NO: 636)
H-G-E-G-T-F-T-S-D-X₁₀-S-K-Q-L-E-E-E-X₁₈-V-X₂₀-L-F-I-
E-W-L-K-A-X₂₉-G,

wherein

### X₁₀ is K or L,

### X₁₈ is A or R,

### X₂₀ is R or Q, and

### X₂₉ is G or T;

wherein, optionally, the amino acid sequence further comprises at least one additional amino acid residue at its N-terminus; and
wherein, optionally, the amino acid sequence further comprises a peptide extension consisting of up to about 12, about 11 or about 10 amino acid residues at its C-terminus.

In one embodiment, the at least one additional amino acid residue is G or A. In one embodiment, the at least one additional amino acid residue is a single amino acid residue. In one embodiment, the at least one additional amino acid residue is G.

In one embodiment, the peptide extension comprises or consists of an amino acid sequence selected from the group consisting of SEQ ID NOs: 566 to 621. In one embodiment, the peptide extension is a single amino acid residue, e.g., P. In one embodiment, the peptide extension comprises or consists of the amino acid sequence PSSGAPPPS (SEQ ID NO: 605) or PKKIRYS (SEQ ID NO: 598).

In another aspect, the present invention relates to a GLP-1R agonistic peptide having a GLP-1R agonistic activity which is about 9- to about 531-fold reduced as compared to the GLP-1R agonistic activity of native GLP-1(7-36) (SEQ ID NO: 260), wherein the GLP-1R agonistic peptide comprises or consists of an amino acid sequence selected from the group consisting of SEQ ID NOs: 261 to 552 and 554 to 565, or an amino acid sequence selected from the group consisting of SEQ ID NOs: 261 to 552 and 554 to 565, with up to three substitutions of amino acid residues.

In one embodiment, the GLP-1R agonistic peptide has a GLP-1R agonistic activity which is about 9- to about 531-fold reduced as compared to the GLP-1R agonistic activity of native GLP-1(7-36) (SEQ ID NO: 260) when the GLP-1R agonistic peptide is in its isolated form and/or when the GLP-1R agonistic peptide is part of a fusion molecule.

In one embodiment, the GLP-1R agonistic peptide has a GLP-1R agonistic activity which is about 9- to about 482-fold (or about 9.449- to about 482.396-fold) or about 9- to about 319-fold (or about 9.449- to about 319.311-fold) or about 9- to about 121-fold (or about 9.449- to about 121.189-fold) reduced as compared to the GLP-1R agonistic activity of native GLP-1(7-36).

In one embodiment, the GLP-1R agonistic peptide has a GLP-1R agonistic activity which is about 9- to about 319-fold reduced as compared to the GLP-1R agonistic activity of native GLP-1(7-36).

In one embodiment, the GLP-1R agonistic peptide has a GLP-1R agonistic activity which is at least about 9.4-fold or at least about 9.45-fold or at least about 9.5-fold reduced as compared to the GLP-1R agonistic activity of native GLP-1(7-36).

In one embodiment, the GLP-1R agonistic peptide has a GLP-1R agonistic activity which is at least about 10-fold reduced as compared to the GLP-1R agonistic activity of native GLP-1(7-36).

In one embodiment, the GLP-1R agonistic peptide has a GLP-1R agonistic activity which is at most about 482.4-fold or at most about 482.35-fold reduced as compared to the GLP-1R agonistic activity of native GLP-1(7-36).

In one embodiment, the GLP-1R agonistic peptide has a GLP-1R agonistic activity which is at most about 482-fold reduced as compared to the GLP-1R agonistic activity of native GLP-1(7-36).

In one embodiment, the GLP-1R agonistic peptide has a GLP-1R agonistic activity which is about 10- to about 482-fold reduced as compared to the GLP-1R agonistic activity of native GLP-1(7-36).

In one embodiment, the GLP-1R agonistic peptide has a GLP-1R agonistic activity which is about 10- to about 319-fold reduced as compared to the GLP-1R agonistic activity of native GLP-1(7-36).

In one embodiment, the GLP-1R agonistic peptide has a GLP-1R agonistic activity which is about 90- to about 100-fold reduced as compared to the GLP-1R agonistic activity of native GLP-1(7-36).

In one embodiment, the GLP-1R agonistic peptide has a GLP-1R agonistic activity which is at least about 18-fold (or at least about 18.268-fold) reduced as compared to the GLP-1R agonistic activity of native GLP-1(7-36).

In one embodiment, the GLP-1R agonistic peptide has a GLP-1R agonistic activity which is about 18- to about 501-fold (or about 18.268- to about 500.686-fold) or about 18- to about 469-fold (or about 18.268- to about 468.679-fold) or about 18- to about 313-fold (or about 18.268- to about 313.214-fold) or about 18- to about 123-fold (or about 18.268- to about 123.466-fold) reduced as compared to the GLP-1R agonistic activity of native GLP-1(7-36).

In one embodiment, the GLP-1R agonistic peptide has a GLP-1R agonistic activity which is about 18- to about 313-fold reduced as compared to the GLP-1R agonistic activity of native GLP-1(7-36).

In one of the above embodiments, the GLP-1R agonistic peptide has a GLP-1R agonistic activity which is at least about 18.2-fold or at least about 18.3-fold reduced as compared to the GLP-1R agonistic activity of native GLP-1(7-36).

In one of the above embodiments, the GLP-1R agonistic peptide has a GLP-1R agonistic activity which is at least about 20-fold or at least about 50-fold or at least about 100-fold reduced as compared to the GLP-1R agonistic activity of native GLP-1(7-36).

In one embodiment, the GLP-1R agonistic peptide has a GLP-1R agonistic activity which is about 10-fold to about 500-fold reduced as compared to the GLP-1R agonistic activity of native GLP-1(7-36). In one embodiment, the GLP-1R agonistic peptide has a GLP-1R agonistic activity which is about 15-fold to about 500-fold reduced as compared to the GLP-1R agonistic activity of native GLP-1(7-36). In one embodiment, the GLP-1R agonistic peptide has a GLP-1R agonistic activity which is about 20-fold to about 500-fold reduced as compared to the GLP-1R agonistic activity of native GLP-1(7-36). In one embodiment, the GLP-1R agonistic peptide has a GLP-1R agonistic activity which is about 50-fold to about 500-fold reduced as compared to the GLP-1R agonistic activity of native GLP-1(7-36). In one embodiment, the GLP-1R agonistic peptide has a GLP-1R agonistic activity which is about 100-fold to about 500-fold reduced as compared to the GLP-1R agonistic activity of native GLP-1(7-36). In one embodiment, the GLP-1R agonistic peptide has a GLP-1R agonistic activity which is about 100-fold to about 300-fold reduced as compared to the GLP-1R agonistic activity of native GLP-1(7-36).

In one embodiment, the GLP-1R agonistic peptide as part of a fusion molecule activates human GLP-1R with an EC50 of about 15 pmol/L to about 400 pmol/L, or about 20 pmol/L to about 400 pmol/L, or about 50 pmol/L to about 400 pmol/L, or about 100 pmol/L to about 400 pmol/L, as determined, e.g., by measuring the cAMP response of cells stably expressing human GLP-1R. In one embodiment, activation of human GLP-1R is determined essentially as described in Example 4.

In one embodiment, the GLP-1R agonistic peptide in its isolated form activates human GLP-1R with an EC50 of about 7.5 pmol/L to about 250 pmol/L, or about 7.5 pmol/L to about 150 pmol/L, or about 7.5 pmol/L to about 100 pmol/L, or about 7.5 pmol/L to about 75 pmol/L, or about 8 pmol/L to about 75 pmol/L, or 9 about pmol/L to 75 about pmol/L, or 9 about pmol/L to 60 about pmol/L, as determined, e.g., by measuring the cAMP response of cells stably expressing human GLP-1R. In one embodiment, activation of human GLP-1R is determined essentially as described in Example 4.

In one embodiment, the GLP-1R agonistic peptide comprises or consists of an amino acid sequence selected from the group comprising or consisting of SEQ ID NOs: 261 to 552 and 554 to 565.

In one embodiment, the GLP-1R agonistic peptide comprises or consists of the amino acid sequence of SEQ ID NO: 261 or 262.

In one embodiment, the GLP-1R agonistic peptide does not comprise or consist of an amino acid sequence of any one of SEQ ID NOs: 553 and 622 to 634.

In another aspect, the present invention relates to a GLP-1R agonistic peptide comprising or consisting of an amino acid sequence selected from the group consisting of SEQ ID NOs: 261 to 552 and 554 to 565.

In another aspect, the present invention relates to a GLP-1R agonistic peptide comprising or consisting of an amino acid sequence of SEQ ID NO: 261 or comprising or consisting of an amino acid sequence of SEQ ID NO: 262 In another aspect, the present invention relates to a combination comprising a GLP-1R agonistic peptide as defined above and at least one other active pharmaceutical ingredient.

In another aspect, the present invention relates to a fusion molecule comprising a GLP-1R agonistic peptide as defined above and at least one other active pharmaceutical ingredient.

In one embodiment, the at least one other active pharmaceutical ingredient is an FGF21 compound.

In another aspect, the present invention relates to a nucleic acid molecule encoding a GLP-1R agonistic peptide as defined above or a fusion molecule as defined above.

In another aspect, the present invention relates to a host cell containing a nucleic acid molecule as defined above.

In another aspect, the present invention relates to a pharmaceutical composition comprising a GLP-1R agonistic peptide as defined above, a combination as defined above, a fusion molecule as defined above, a nucleic acid molecule as defined above or a host cell as defined above.

In another aspect, the present invention relates to a kit comprising a GLP-1R agonistic peptide as defined above, a combination as defined above, a fusion molecule as defined above, a nucleic acid molecule as defined above, a host cell as defined above or a pharmaceutical composition as defined above.

In another aspect, the present invention relates to a GLP-1R agonistic peptide as defined above, a combination as defined above, a fusion molecule as defined above, a nucleic acid molecule as defined above, a host cell as defined above or a pharmaceutical composition as defined above for use as a medicament.

In another aspect, the present invention relates to a GLP-1R agonistic peptide as defined above, a combination as defined above, a fusion molecule as defined above, a nucleic acid molecule as defined above or a host cell as defined above or a pharmaceutical composition as defined above for use in the treatment of a disease or disorder selected from the group consisting of obesity, being overweight, metabolic syndrome, diabetes mellitus, hyperglycemia, dyslipidemia, NASH and atherosclerosis.

In one embodiment, the disease or disorder is diabetes mellitus. In one embodiment, the diabetes mellitus is type 1 diabetes mellitus or type 2 diabetes mellitus.

In another aspect, the present invention relates to the use of a GLP-1R agonistic peptide as defined above, a combination as defined above, a fusion molecule as defined above, a nucleic acid molecule as defined above, a host cell as defined above or a pharmaceutical composition as defined above in the manufacture of a medicament for the treatment of a disease or disorder selected from the group consisting of obesity, being overweight, metabolic syndrome, diabetes mellitus, diabetic retinopathy, hyperglycemia, dyslipidemia, NASH and atherosclerosis.

In one embodiment, the disease or disorder is diabetes mellitus. In one embodiment, the diabetes mellitus is type 1 diabetes mellitus or type 2 diabetes mellitus.

In another aspect, the present invention relates to a method of treating a disease or disorder selected from the group consisting of obesity, being overweight, metabolic syndrome, diabetes mellitus, diabetic retinopathy, hyperglycemia, dyslipidemia, NASH and atherosclerosis, the method comprising administering a GLP-1R agonistic peptide as defined above, a combination as defined above, a fusion molecule as defined above, a nucleic acid molecule as defined above, a host cell as defined above or a pharmaceutical composition as defined above to a subject in need thereof.

In one embodiment, the disease or disorder is diabetes mellitus. In one embodiment, the diabetes mellitus is type 1 diabetes mellitus or type 2 diabetes mellitus.

### DESCRIPTION OF THE FIGURES

FIG. 1 is a graph showing EC50 of the adverse effect (Gastric Emptying (GE) rate) and pharmacodynamics (i.e., HbA1c, Triglycerides, Fatty Acids, Non-HDL, Adipose Mass) depending on the GLP-1 attenuation factor (12-months simulation):
For GLP-1 attenuation factors greater than 9.449 (which can be rounded to about 9), the EC50 of GLP-1-mediated gastrointestinal adverse effect (gastric emptying; GE-Rate) was greater than EC50 of pharmacodynamic effects (i.e., HbA1c, Adipose Mass, Non-HDL, Fatty Acids, Triglycerides);
The maximal distance between maximum of pharmacodynamics (HbA1c) and adverse effect (GE rate) normalized by spreading of FGF21- (lipids) and GLP-1-mediated effects (HbA1c) was 121.189; i.e., at 121.189 (which can be rounded to about 121), there is a maximal distance between maximum of pharmacodynamics effects (HbA1c) and adverse effect (GE rate) at a minimum distance between GLP-1-mediated effects (HbA1c) and mean FGF21-mediated effects (i.e., Adipose Mass, Non-HDL, Fatty Acids, Triglycerides) (see FIG. 2);
The maximal distance between maximum of pharmacodynamics (HbA1c) and adverse effect (GE rate) was 319.311 (which can be rounded to about 319);
The maximal distance between mean pharmacodynamics (i.e., HbA1c, Adipose Mass, Non-HDL, Fatty Acids, Triglycerides) and adverse effect (GE rate) was 482.396 (see FIG. 2; which can be rounded to about 482);
The maximum gastric emptying rate was 531.0;
   (all: vertical lines).

FIG. 2 is a graph showing the EC50 for GE rate and mean pharmacodynamic effects (i.e., HbA1c, Triglycerides, Fatty Acids, Non-HDL, Adipose Mass) depending on GLP-1 attenuation factor (12-months simulation):
The maximal distance between mean pharmacodynamics (i.e., HbA1c, Adipose Mass, Non-HDL, Fatty Acids, Triglycerides) and adverse effect (GE-Rate) was 482.396 (right vertical line; which can be rounded to about 482);
The maximal distance between maximum of pharmacodynamics (HbA1c) and adverse effect (GE rate) normalized by spreading of FGF21- (lipids) and GLP-1-mediated effects (HbA1c) was 121.189 (left vertical line; which can be rounded to about 121). The curve “(Max-GE Rate)/Range” represents the ratio between the maximum distance between HbA1c and GE rate and the minimum distance between HbA1c and mean FGF21-mediated effects (i.e., Adipose Mass, Non-HDL, Fatty Acids, Triglycerides). At the minimum of the “(Max-GE Rate)/Range” curve (i.e., at 121.189), there is a maximal distance between maximum of pharmacodynamics effects (HbA1c) and adverse effect (GE rate) at a minimum distance between GLP-1-mediated effects (HbA1c) and FGF21-mediated effects (i.e., Adipose Mass, Non-HDL, Fatty Acids, Triglycerides).

FIG. 3 is a graph showing EC50 of the adverse effect (GE rate) and pharmacodynamics (HbA1c, Triglycerides, Fatty Acids, Non-HDL, Adipose Mass) depending on GLP-1 attenuation factor (3-months simulation):
For GLP-1 attenuation factors greater than 18.268 (which can be rounded to about 18), the EC50 of GLP-1-mediated gastrointestinal adverse effect (gastric emptying; GE-Rate) was greater than the EC50 of pharmacodynamic effects (i.e., HbA1c, Adipose Mass, Non-HDL, Fatty Acids, Triglycerides);
The maximal distance between maximum of pharmacodynamics (HbA1c) and adverse effect (GE rate) normalized by spreading of FGF21- (lipids) and GLP-1-mediated effects (HbA1c) was 123.466; i.e., at 123.466 (which can be rounded to about 123), there is a maximal distance between maximum of pharmacodynamics effects (HbA1c) and adverse effect (GE rate) at a minimum distance between GLP-1-mediated effects (HbA1c) and mean FGF21-mediated effects (i.e., Adipose Mass, Non-HDL, Fatty Acids, Triglycerides) (see FIG. 4);
The maximal distance between maximum of pharmacodynamics (HbA1c) and adverse effect (GE-Rate) was 313.214 (which can be rounded to about 313);
The maximal distance between mean pharmacodynamics (i.e., HbA1c, Adipose Mass, Non-HDL, Fatty Acids, Triglycerides) and adverse effect (GE-Rate) was 468.679 (see FIG. 4; which can be rounded to about 469);
The maximum GE rate was at 500.686 (which can be rounded to about 501) (all: vertical lines).

FIG. 4 is a graph showing the EC50 of GE rate and mean pharmacodynamic effects (i.e., HbA1c, Triglycerides, Fatty Acids, Non-HDL, Adipose Mass) depending on GLP-1 attenuation factor (3-months simulation):
The maximal distance between mean pharmacodynamics (i.e., HbA1c, Adipose Mass, Non-HDL, Fatty Acids, Triglycerides) and adverse effect (GE-Rate) was 468.679 (right vertical line; which can be rounded to about 469);
The maximal distance between maximum of pharmacodynamics (HbA1c) and adverse effect (GE rate) normalized by spreading of FGF21- (lipids) and GLP-1-mediated effects (HbA1c) was 123.466 (left vertical line; which can be rounded to about 123). The curve “(Max-GE Rate)/Range” represents the ratio between the maximum distance between HbA1c and GE rate and the minimum distance between HbA1c and mean FGF21-mediated effects (i.e., Adipose Mass, Non-HDL, Fatty Acids, Triglycerides). At the minimum of the “(Max-GE Rate)/Range” curve (i.e., at 123.466), there is a maximal distance between maximum of pharmacodynamics effects (HbA1c) and adverse effect (GE rate) at a minimum distance between GLP-1-mediated effects (HbA1c) and FGF21-mediated effects (i.e., Adipose Mass, Non-HDL, Fatty Acids, Triglycerides).

FIGS. 5 (A and B) are graphs showing the results of an in vitro cellular assay (In-Cell Western (ICW)) for human FGF21 receptor efficacy in CHO cells. pFGFR is depicted in (A), and pERK is depicted in (B).

FIGS. 6 (A to D) are graphs showing the results of an in vitro cellular assay for human Glucagon-Like-Peptide 1 (GLP-1) receptor efficacy in HEK-293 cells for different GLP-1R agonists. SEQ ID NO: 2 is depicted in (A), SEQ ID NO: 7 is depicted in (B), SEQ ID NO: 8 is depicted in (C), and SEQ ID NOs: 2, 7, and 8 are depicted in (D).

FIGS. 7 (A to F) are graphs showing plasma concentrations of GLP-1R agonist/FGF21 Fc fusion proteins after single subcutaneous administration of a 0.3 mg/kg solution to female C57BI/6 mice or male cynomolgus monkeys using three different bioanalytical methods. (A) depicts SEQ ID NO: 2 in mouse, (B) depicts SEQ ID NO: 2 in monkey, (C) depicts SEQ ID NO: 7 in mouse, (D) depicts SEQ ID NO: 7 in monkey, (E) depicts SEQ ID NO: 8 in mouse, and (F) depicts SEQ ID NO: 8 in monkey.

FIG. 8 is a graph showing shows plasma concentrations of GLP-1R agonist/FGF21 Fc fusion proteins and G-FGF21 (SEQ ID NO: 252) after single subcutaneous administration of a 0.3 mg/kg solution to female C57BI/6 mice using a bioanalytical method for quantification of the intact full-length proteins.

FIG. 9 is a graph showing the development of body weight in female Diet-Induced Obesity (DIO) mice with once weekly dosing of GLP-1RA/FGF21 Fc fusion proteins and controls for 28 days.

FIG. 10 is a graph showing the development of cumulative food intake in female DIO mice with once weekly dosing of GLP-1 RA/FGF21 Fc fusion proteins and controls for 28 days.

FIGS. 11 (A and B) are graphs showing the 24 hour blood glucose profile of db/db mice following the first treatment with GLP-1RA/FGF21 Fc fusion proteins and controls starting on day 1 (A) or following the fourth treatment starting on day 22 (B). Data are means±SEM; n=8/group.

FIG. 12 is a graph showing the plasma HbA1c content in female db/db mice with a once weekly dosing of GLP-1RA/FGF21 Fc fusion proteins and controls for 36 days.

FIGS. 13 (A and B) are graphs showing the development of liver weight and lipid contents in DIO NASH mice after once weekly dosing of a GLP-1RA/FGF21 Fc fusion protein and controls for 8 weeks. (A) depicts liver weight and lipid levels, (B) depicts liver cholesterol and liver triglyceride levels.

FIG. 14 depicts graphs showing the development of the development of fibrosis and Non-Alcoholic Fatty Liver Disease (NAFLD) activity score in DIO NASH mice after once weekly dosing of a GLP-1 RA/FGF21 Fc fusion protein and controls for 8 weeks.

FIG. 15 depicts graphs showing the number of animals with higher, the same or lower fibrosis and NAFLD activity score in DIO NASH mice after once weekly dosing of a GLP-1RA/FGF21 Fc fusion protein and controls for 8 weeks.

### DETAILED DESCRIPTION OF THE INVENTION

Although the present invention is described in detail below, it is to be understood that this invention is not limited to the particular methodologies, protocols and reagents described herein, as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

Certain elements of the present invention will be described in more detail herein. These elements may be listed with specific embodiments; however, it should be understood that particular specific embodiments may be combined in any manner and in any number to create additional embodiments. The variously described examples and exemplary embodiments should not be construed to limit the present invention to only the explicitly described embodiments. This description should be understood to support and encompass embodiments which combine the explicitly described embodiments with any number of the disclosed and/or exemplary elements. Furthermore, any permutations and combinations of all described elements in this application should be considered disclosed by the description of the present application unless the context indicates otherwise.

The terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present invention will employ, unless otherwise indicated, conventional methods of chemistry, biochemistry, cell biology, immunology, and recombinant DNA techniques which are explained in the literature in the field (Sambrook, J. et al. (2001) Molecular Cloning: A Laboratory Manual, 3rd Ed., Cold Spring Harbor Laboratory Press, Cold Spring Harbor, N.Y.).

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e., the subject-matter consists in the inclusion of a stated member, integer or step or group of members, integers or steps. The terms “a” and “an” and “the” and and the like, used in the context of describing the invention (especially in the context of the claims), are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Several documents are cited throughout the text of this specification. Each of the documents cited herein (including all patents, patent applications, scientific publications, manufacturer's specifications, instructions, etc.), whether supra or infra, is hereby incorporated by reference in its entirety. Nothing herein is to be construed as an admission that the invention is not entitled to antedate such disclosure by virtue of prior invention.

The term “GLP-1R agonistic peptide”, as used herein, refers to a peptide which binds to and activates the GLP-1 receptor, such as GLP-1 (as the primary GLP-1R agonist). GLP-1R agonistic peptides may also be simply referred to as “GLP-1R agonists” herein.

The term “peptide”, as used herein, generally refers to a polymeric form of amino acids of any length, for example, comprising about two or more, or about 3 or more, or about 4 or more, or about 6 or more, or about 8 or more, or about 9 or more, or about 10 or more, or about 13 or more, or about 16 or more, or about 21 or more amino acids joined covalently by peptide bonds. A peptide may, for example, consist of up to about 100 amino acids. The term “polypeptide”, as used herein, refers to large peptides. In one embodiment, the term “polypeptide” refers to peptides with more than about 100 amino acid residues. The terms “polypeptide” and “protein” are used interchangeably herein.

The term “amino acid” or “amino acid residue”, as used herein, refers to a naturally occurring amino acid, an unnatural amino acid, an amino acid analogue and/or an amino acid mimetic that functions in a manner similar to the naturally-occurring amino acid in its D or L stereoisomer if its structure allows such stereoisomeric forms. Amino acids are referred to herein by either their name, their art-known three letter symbols or by the one-letter symbols recommended by the IUPAC-IUB Biochemical Nomenclature Commission.

The term “naturally occurring”, as used herein, when used in connection with biological materials such as nucleic acid molecules, (poly-)peptides, host cells, and the like, refers to materials which are found in nature and are not manipulated by man.

When used in connection with amino acids, the term “naturally occurring” refers to the 20 conventional amino acids (i.e., alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y)), as well as selenocysteine, pyrrolysine (PYL), and pyrroline-carboxylysine (PCL).

The term “unnatural amino acid”, as used herein, is meant to refer to an amino acids that is not naturally encoded or found in the genetic code of any organism. It may, for example, be a purely synthetic compound. Examples of unnatural amino acids include, but are not limited to, hydroxyproline, gamma-carboxyglutamate, O-phosphoserine, azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, tertiary-butylglycine, 2,4-diaminoisobutyric acid, desmosine, 2,2′-diaminopimelic acid, 2,3-diaminoproprionic acid, N-ethylglycine, N-methylglycine, N-ethylasparagine, homoproline, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, norleucine, ornithine, D-ornithine, D-arginine, p-aminophenylalanine, pentylglycine, pipecolic acid and thioproline.

The term “amino acid analogue”, as used herein, refers to a compound that has the same basic chemical structure as a naturally occurring amino acid. Amino acid analogues include the natural and unnatural amino acids which are chemically blocked, reversibly or irreversibly, or chemically modified, e.g., at one or any combination of their C-terminal carboxy group, their N-terminal amino group and/or their side-chain functional groups. Such analogues include, but are not limited to, methionine sulfoxide, methionine sulfone, S-(carboxymethyl)-cysteine, S-(carboxymethyl)-cysteine sulfoxide, S-(carboxymethyl)-cysteine sulfone, aspartic acid-(betamethylester), N-ethylglycine, alanine carboxamide, homoserine, norleucine and methionine methyl sulfonium.

The term “amino acid mimetic”, as used herein, refers to a chemical compound that has a structure that is different from the general chemical structure of an amino acid, but functions in a manner similar to a naturally-occurring amino acid.

The term “native GLP-1(7-36)”, as used herein, refers to a peptide having the amino acid sequence of SEQ ID NO: 260, which, optionally, comprises an amide group at its C-terminus.

In one embodiment, the term “GLP-1R agonistic activity” (or “GLP-1R agonistic potency”), as used herein, refers to the activation of the GLP-1 receptor. In one embodiment, the term refers to the agonistic activity/potency in vitro. In another embodiment, the term refers to the agonistic activity/potency in vivo. In one embodiment, activation of the GLP-1 receptor is determined by measuring the cAMP response of cells stably expressing GLP-1 receptor upon contact with the agonist in vitro. In one embodiment, the cells are from a HEK-293 cell line. In one embodiment, the GLP-1 receptor is human GLP-1 receptor. In one embodiment, activation of the GLP-1 receptor is determined essentially as described in Example 4. In one embodiment, the activity/potency is quantified by determining the EC50 value.

The present invention provides GLP-1R agonistic peptides according to the general formulae of SEQ ID NOs: 635 and 636 as defined herein.

In some embodiments, the GLP-1R agonistic peptides according to the general formulae of SEQ ID NOs: 635 and 636 comprise at least one additional amino acid residue at their N-terminus. In one embodiment, the at least one additional amino acid residue is a single amino acid residue. In one embodiment, the at least one additional amino acid residue is selected from the group consisting of: naturally occurring amino acids except proline; unnatural amino acids; amino acid analogues; and amino acid mimetics. In one embodiment, the at least one additional amino acid residue is selected from the group consisting of G, A, N and C. In one embodiment, the at least one additional amino acid residue is G or A. In one embodiment, the at least one additional amino acid residue is G.

In some embodiments, GLP-1R agonistic peptides according to the general formulae of SEQ ID NOs: 635 and 636 comprise a peptide extension at their C-terminus. The peptide extension may, for example, consist of up to about 12, about 11, about 10 or about 9 amino acid residues (e.g., about 7, about 8 or about 9 amino acid residues). In one embodiment, the peptide extension consists of an amino acid sequence selected from the group consisting of SEQ ID NOs: 566 to 621. In one embodiment, the peptide extension is a single amino acid residue, e.g., P.

The present invention also provides GLP-1R agonistic peptides comprising or consisting of an amino acid sequence selected from the group consisting of SEQ ID NOs: 261 to 552 and 554 to 565 as well as variants of these amino acid sequences which differ from the original sequences in the substitution of one, two or three amino acid residues.

The present invention also provides GLP-1R agonistic peptides comprising or consisting of an amino acid sequence that is SEQ ID NO: 261 or SEQ ID NO: 262 as well as variants of these amino acid sequences which differ from the original sequences in the substitution of one, two or three amino acid residues.

In one embodiment, the substituted amino acid residue(s) is/are not involved in the GLP-1R agonistic activity of the GLP-1R agonistic peptide. In one embodiment, the substitution(s) is/are functionally and/or phenotypically silent. In one embodiment, the substitution(s) is/are (a) conservative amino acid substitution(s).

As used herein, the term “conservative amino acid substitution” refers to a substitution of one or more amino acids with one or more amino acids of the same family of amino acids, i.e., amino acids which are related in their side chains (e.g., in terms of the electrical charge and/or size). Naturally occurring amino acids are generally divided into four families: acidic (aspartate, glutamate); basic (lysine, arginine, histidine); non-polar (alanine, valine, leucine, isoleucine, proline, phenylalanine, methionine, tryptophan); and uncharged polar (glycine, asparagine, glutamine, cysteine, serine, threonine, tyrosine). Phenylalanine, tryptophan, and tyrosine are sometimes classified jointly as aromatic amino acids.

The GLP-1R agonistic peptides may be fused or conjugated to a half-life extension module. “Half-life”, as used herein, generally refers to the period of time which is needed to eliminate half of the activity of a compound, the amount of a compound, or number of molecules, e.g., in vivo. Such modules are known to a person skilled in the art and include, for example, polymers (e.g., PolyEthylene Glycol (PEG), HydroxyEthyl Starch (HES), hyaluronic acid, polysialic acid), unstructured (poly-)peptide chains, Elastin-Like Polypeptides (ELPs), serum proteins (e.g., albumin, such as Human Serum Albumin (HAS)), serum protein binding molecules (e.g., an Albumin Binding Domain (ABD), an albumin binding fatty acid), antibodies, immunoglobulins, Fc domains (also referred to as Fc regions) of immunoglobulins and immunoglobulin binding domains.

The term “unstructured (poly-)peptide chain”, as used herein, refers to a (poly-)peptide chain which lacks a fixed or ordered three-dimensional structure and is typically hydrophilic. Unstructured (poly-)peptide chains that extend the half-life (e.g., the in vivo half-life) of peptides and proteins they are fused to are known to a person skilled in the art and include, for example, XTEN (Schellenberger V. et al. (2009) Nat Biotechnol. 27(12): 1186-90) and PAS sequences (Schlapschy M. et al. (2013) Protein Eng Des Sel. 26(8): 489-501).

The term “fused to”, as used herein, refers, in particular, to genetic fusion, e.g., by recombinant DNA technology. The amino acid sequence of a (poly)peptide half-life extension module may be introduced at any position within the amino acid sequence of the GLP-1R agonistic peptide, and may, for example, take the shape of a loop within the encoded peptide structure, or it may be N-terminally or C-terminally fused. The amino acid sequence of the (poly)peptide half-life extension module fused to the GLP-1R agonistic peptide may be encoded by a polynucleotide.

The term “conjugated to”, as used herein, refers to chemical and/or enzymatic conjugation resulting in a stable covalent link between a (poly-)peptide and another molecule, e.g., the GLP-1R agonistic peptide and the half-life extension module. Such conjugation may occur at the N- or C-terminus or at particular side chains of a (poly-)peptide, e.g., at lysine, cysteine, tyrosine or an unnatural amino acid residue.

The term “combination”, as used herein, is meant to include means that allow to apply the combination comprising the GLP-1R agonistic peptide and the at least one other active pharmaceutical ingredient either by separate administration of the GLP-1R agonistic peptide and the at least one other active pharmaceutical ingredient to the patient or in the form of combination products in which the GLP-1R agonistic peptide and the at least one other active pharmaceutical ingredient are present, e.g., in one pharmaceutical composition or in the form of a fusion molecule. When administered separately, administration may occur simultaneously or sequentially, in any order. The amount of the GLP-1R agonistic peptide and the at least one other active pharmaceutical ingredient as well as the relative timings of administration will be selected in order to achieve the desired combined therapeutic effect. The administration of the combination may be concomitantly in: (1) a unitary pharmaceutical composition including all active pharmaceutical ingredients; or (2) separate pharmaceutical compositions each including at least one of the active pharmaceutical ingredients. Alternatively, the combination may be administered separately in a sequential manner wherein one treatment agent is administered first and the other second or vice versa. Such sequential administration may be close in time or remote in time. In one embodiment, the combination is provided in the form of a kit, e.g., a kit as defined herein.

The term “fusion molecule”, as used herein, generally refers to a molecule created by joining, in particular covalently linking, two or more distinct molecules (e.g., proteins and/or peptides and/or combinations thereof) resulting in the formation of a single molecule. In certain exemplary embodiments, the fusion molecule will have one or more functional properties derived from each of the original molecules. In the case of proteins and/or peptides, the fusion molecule is also referred to as “fusion protein”. Fusion molecules may be generated by genetic fusion (e.g., by recombinant DNA technology) or by chemical and/or enzymatic conjugation, e.g., of two or more polypeptide, proteins, or any combination thereof. The two or more distinct molecules may also be linked by one or more suitable linker molecules, e.g., peptide linkers and/or non-peptidic polymers, such as PEG.

In one embodiment, peptide linkers have a length of about 2 to about 100 amino acid residues, or about 2 to about 90 amino acid residues, or about 2 to about 80 amino acid residues, or about 2 to about 70 amino acid residues, or about 2 to about 60 amino acid residues, or about 2 to about 50 amino acid residues, or about 2 to about 40 amino acid residues, or about 2 to about 30 amino acid residues, or about 2 to about 25 amino acid residues, or about 2 to about 20 amino acid residues. In one embodiment, the peptide linker comprises at least about 5 amino acid residues. In general, peptide linkers are designed to provide flexibility and protease resistance. In one embodiment, the peptide linker is a glycine-serine-rich linker, wherein, e.g., at least about 50%, at least about 60%, at least about 70%, at least about 80%, at least about 85% of the amino acids are a glycine or serine residue, respectively. In another embodiment, the amino acids are selected from glycine and serine, i.e., the peptide linker is exclusively composed of glycine and serine (referred to as a glycine-serine linker). In one embodiment, the peptide linker further comprises an alanine residue at its C-terminus. Peptide linkers may further comprise one or more specific protease cleavage sites. In one embodiment, the peptide linker comprises or consists of an amino acid sequence selected from the group consisting of SEQ ID NOs: 231 to 245.

In one embodiment, a fusion molecule further comprises an Fc domain (also referred to as Fc region) of an immunoglobulin (e.g., IgG1 or IgG4) or a variant thereof. In one embodiment, the variant of the Fc domain comprises up to about 6, about 5 or about 4 mutations as compared to the wild-type sequence of the Fc domain. In one embodiment, said mutations are selected from the group consisting of amino acid substitutions, amino acid additions and amino acid deletions, e.g., N- or C-terminal deletions. In one embodiment, the Fc domain or variant thereof can have greater than about 50%, about 60%, about 70%, about 80%, about 90%, about 93%, about 95%, about 96%, about 97%, about 98% or about 99% sequence identity or can have about 100% sequence identity to the wild-type sequence of the IgG1 Fc region, e.g., the human IgG1 Fc region. In one embodiment, the Fc domain or variant thereof can have greater than about 50%, about 60%, about 70%, about 80%, about 90%, about 93%, about 95%, about 96%, about 97%, about 98% or about 99% sequence identity or can have about 100% sequence identity to the wild-type sequence of the IgG4 Fc region, e.g., the human IgG4 Fc region. In one embodiment, the Fc domain of an immunoglobulin or a variant thereof comprises or consists of an amino acid sequence selected from the group consisting of SEQ ID NOs: 257, 258 and 259.

In one embodiment, a fusion molecule comprises a linker molecule comprising a structure selected from the group consisting of L-Fc, Fc-L, L₁-Fc-L₂ and Fc, wherein L, L₁ and L₂ are independently selected from the group consisting of single amino acids and peptides (e.g., peptide linkers as defined herein), and Fc is an Fc domain of an immunoglobulin or a variant thereof. L₁ and L₂ may be the same or different. In one embodiment, L₁ and L₂ are different. In one embodiment, L₁ comprises or consists of the amino acid sequence of SEQ ID NO: 232, and L₂ comprises or consists of the amino acid sequence of SEQ ID NO: 231, or vice versa.

The phrase “fibroblast growth factor 21” or “FGF21”, as used herein, refers to any FGF21 protein known in the art and particularly refers to human FGF21. In one embodiment, human FGF21 has the amino acid sequence of SEQ ID NO: 250 (full-length human wild-type FGF21). Mature human wild-type FGF21, i.e., a human wild-type FGF21 lacking amino acids 1 to 28 (M1 to A28) of SEQ ID NO: 250 (i.e., its signal sequence/peptide), is set forth as SEQ ID NO: 251. Mature human wild-type FGF21 with an additional N-terminal Gly is represented by SEQ ID NO: 252 and is referred to herein as G-FGF21.

The phrase “FGF21 compound”, as used herein, generally refers to a compound having FGF21 activity.

In one embodiment, the phrase “FGF21 activity” (or “FGF21 potency”), as used herein, refers to activation of the FGF21 receptor (FGFR, e.g., FGFR1c). In one embodiment, the FGF21 receptor is a human FGF21 receptor. In one embodiment, FGF21 activity refers to activity and/or potency in vitro. In another embodiment, FGF21 activity refers to activity and/or potency in vivo. In one embodiment, activation of the FGF21 receptor is determined by measuring FGF21 receptor autophosphorylation and/or phosphorylation of MAPK ERK1/2 upon contact with the FGF21 compound in vitro. In one embodiment, autophosphorylation of human FGFR1c and/or phosphorylation of MAPK ERK1/2 is/are determined by using an In-Cell Western (ICW), e.g., essentially as described in Example 3. In one embodiment, the activity and/or potency is quantified by determining the EC50 value.

The term “In-Cell Western (ICW) assay”, as used herein, refers to an immunocytochemical assay, more particularly a quantitative immunofluorescence assay that is typically performed using microplates (e.g., in a 96- or 384-well format). ICW combines the specificity of Western blotting with the reproducibility and throughput of ELISA (see, for example, Aguilar H. N. et al. (2010) PLoS ONE 5(4): e9965). Appropriate ICW assay systems are commercially available (e.g., from LI-COR Biosciences, USA). In one embodiment, an anti-pFGFR antibody and/or an anti-pERK antibody is/are used in the ICW assay.

In one embodiment, the FGF21 compound is a peptidic compound, i.e., a peptide or protein. In one embodiment, the FGF21 compound is native FGF21 or an FGF21 variant having at least about 80% or at least about 90% or at least about 91% or at least about 92% or at least about 93% or at least about 94% or at least about 95% or at least about 96% or at least about 97% or at least about 98% amino acid sequence identity to the amino acid sequence of native FGF21. The term “native FGF21”, as used herein, refers to a naturally occurring FGF21, e.g., a human wild-type FGF21 with the amino acid sequence of SEQ ID NO: 250 or a mature human wild-type FGF21 with the amino acid sequence of SEQ ID NO: 251.

“Sequence identity”, as used herein, refers to the percentage of amino acids that are identical between two amino acid or nucleic acid sequences. The optimal alignment of the sequences for comparison may be produced manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

In one embodiment, an FGF21 compound is a protein comprising or consisting of an amino acid sequence selected from the group consisting of SEQ ID NOs: 250 to 256.

Suitable FGF21 variants for use in the present invention are also described, e.g., in PCT/EP2016/079551, which is incorporated herein by reference.

In one embodiment, a fusion molecule is a fusion protein having the structure A-L₁-Fc-L₂-B, wherein A is a GLP-1R agonistic peptide, L₁, Fc and L₂ are as defined herein, and B is an FGF21 compound as defined herein. In one embodiment, the fusion molecule is a fusion protein comprising or consisting of an amino acid sequence selected from the group consisting of SEQ ID NOs: 1 to 230.

In general, the term “Active Pharmaceutical Ingredient” (API), as used herein, includes any pharmaceutically active chemical or biological compound and any pharmaceutically acceptable salt thereof and any mixture thereof, that provides some pharmacologic effect and is used for treating or preventing a condition, e.g., a disease or disorder, as defined herein.

Exemplary pharmaceutically acceptable salts include, but are not limited to, salts made from one or more of the following acids: hydrochloric acid (e.g., chloride salts), sulfuric acid (e.g., sulfate salts), nitric acid (e.g., nitrate salts), phosphoric acid (e.g., phosphate salts), hydrobromic acid (e.g., hydrobromide salts), maleic acid (e.g., maleate salts), malic acid (e.g., malate salts), ascorbic acid, citric acid (e.g., citrate salts), tartaric acid (e.g., tartrate salts), pamoic acid (e.g., pamoate or embonate salts), lauric acid (e.g., laurate salts), stearic acid (e.g., stearate salts), palmitic acid (e.g., palmitate slats), oleic acid, myristic acid (e.g., myristate salts), lauryl acid, naphthalinesulfonic acid, linolenic acid (e.g., linoleate salts), and the like.

As used herein, the terms “active pharmaceutical ingredient”, “active agent”, “active ingredient”, “active substance”, “therapeutically active compound” and “drug” are meant to be synonyms, i.e., have identical meaning.

In accordance with the present invention, an active pharmaceutical ingredient is optionally selected from:
all drugs mentioned in the Rote Liste 2014, e.g., all antidiabetics mentioned in the Rote Liste 2014, chapter 12, all weight-reducing agents or appetite suppressants mentioned in the Rote Liste 2014, chapter 06, all lipid-lowering agents mentioned in the Rote Liste 2014, chapter 58, all anti-hypertensives mentioned in the Rote Liste 2014 chapter 17, all nephroprotectives mentioned in the Rote Liste, or all diuretics mentioned in the Rote Liste 2014, chapter 36;
an FGF21 compound as defined herein;
a monoclonal antibody;
insulin and insulin derivatives, for example: insulin glargine (e.g., Lantus®), higher than 100 U/mL concentrated insulin glargine, e.g., 270-330 U/mL of insulin glargine or 300 U/mL of insulin glargine (as disclosed in EP 2387989), insulin glulisine (e.g., Apidra®), insulin detemir (e.g., Levemir®), insulin lispro (e.g., Humalog®, Liprolog®), insulin degludec (e.g., DegludecPlus®, IdegLira (NN9068)), insulin aspart and aspart formulations (e.g., NovoLog®), basal insulin and analogues (e.g., LY2605541, LY2963016, NN1436), PEGylated insulin lispro (e.g., LY-275585), long-acting insulins (e.g., NN1436, Insumera (PE0139), AB-101, AB-102, Sensulin LLC), intermediate-acting insulins (e.g., Humulin®N, Novolin®N), fast-acting and short-acting insulins (e.g., Humulin®R, Novolin®R, Linjeta® (VIAject®), PH20 insulin, NN1218, HinsBet®), premixed insulins, SuliXen®, NN1045, insulin plus Symlin®, PE-0139, ACP-002 hydrogel insulin, and oral, inhalable, transdermal and buccal or sublingual insulins (e.g., Exubera®, Nasulin®, Afrezza®, insulin tregopil, TPM-02 insulin, Capsulin®, Oral-lyn®, Cobalamin® oral insulin, ORMD-0801, Oshadi oral insulin, NN1953, NN1954, NN1956, VIAtab®). Also suitable are those insulin derivatives which are bonded to albumin or another protein by a bifunctional linker;
glucagon-like-peptide 1 (GLP-1), GLP-1 analogues, and GLP-1 receptor agonists, for example: GLP-1(7-37), GLP-1(7-36)amide, lixisenatide (e.g., Lyxumia®), exenatide (e.g., exendin-4, rExendin-4, Byetta®, Bydureon®, exenatide NexP), exenatide-LAR, liraglutide (e.g., Victoza®), semaglutide, taspoglutide, albiglutide, dulaglutide, albugon, oxyntomodulin, geniproside, ACP-003, CJC-1131, CJC-1134-PC, GSK-2374697, PB-1023, TTP-054, langlenatide (HM-11260C), CM-3, GLP-1 Eligen, AB-201, ORMD-0901, NN9924, NN9926, NN9927, Nodexen, Viador-GLP-1, CVX-096, ZYOG-1, ZYD-1, ZP-3022, CAM-2036, DA-3091, DA-15864, ARI-2651, ARI-2255, exenatide-XTEN (VRS-859), exenatide-XTEN+Glucagon-XTEN (VRS-859+AMX-808) and polymer-bound GLP-1 and GLP-1 analogues;
dual GLP-1/GIP agonists (e.g., RG-7697 (MAR-701), MAR-709, BHM081, BHM089, BHM098); dual GLP-1/glucagon receptor agonists (e.g., BHM-034, OAP-189 (PF-05212389, TKS-1225), TT-401/402, ZP2929, LAPS-HMOXM25, MOD-6030);
dual GLP-1/gastrin agonists (e.g., ZP-3022);
gastrointestinal peptides such as peptide YY 3-36 (PYY3-36) or analogues thereof and pancreatic polypeptide (PP) or analogues thereof;
glucagon receptor agonists or antagonists, glucose-dependent insulinotropic polypeptide (GIP) receptor agonists or antagonists, ghrelin antagonists or inverse agonists, xenin and analogues thereof;
dipeptidyl peptidase-IV (DPP-4) inhibitors, for example: alogliptin (e.g., Nesina®, Kazano®), linagliptin (e.g., Ondero®, Trajenta®, Tradjenta®, Trayenta®), saxagliptin (e.g., Onglyza®, Komboglyze XR®), sitagliptin (e.g., Januvia®, Xelevia®, Tesavel®, Janumet®, Velmetia®, Juvisync©, Janumet XR®), anagliptin, teneligliptin (e.g., Tenelia®), trelagliptin, vildagliptin (e.g., Galvus®, Galvumet®), gemigliptin, omarigliptin, evogliptin, dutogliptin, DA-1229, MK-3102, KM-223, KRP-104, PBL-1427, Pinoxacin hydrochloride, and Ar-2243;
sodium-dependent glucose transporter 2 (SGLT-2) inhibitors, for example: canagliflozin, dapagliflozin, remogliflozin, remogliflozin etabonate, sergliflozin, empagliflozin, ipragliflozin, tofogliflozin, luseogliflozin, ertugliflozin, EGT-0001442, LIK-066, SBM-TFC-039, and KGA-3235 (DSP-3235);
dual inhibitors of SGLT-2 and SGLT-1 (e.g., LX-4211, LIK066).
SGLT-1 inhibitors (e.g., LX-2761, KGA-3235) or SGLT-1 inhibitors in combination with anti-obesity drugs such as ileal bile acid transfer (IBAT) inhibitors (e.g., GSK-1614235+GSK-2330672);
biguanides (e.g., metformin, buformin, phenformin);
thiazolidinediones (e.g., pioglitazone, rosiglitazone), glitazone analogues (e.g., lobeglitazone);
Peroxisome Proliferator-Activated Receptors (PPAR-alpha, -gamma or -alpha/gamma) agonists or modulators (e.g., saroglitazar (e.g., Lipaglyn®), GFT-505), or PPAR gamma partial agonists (e.g., Int-131);
sulfonylureas (e.g., tolbutamide, glibenclamide, glimepiride, Amaryl®, glipizide) and meglitinides (e.g., nateglinide, repaglinide, mitiglinide);
alpha-glucosidase inhibitors (e.g., acarbose, miglitol, voglibose);
amylin and amylin analogues (e.g., pramlintide, Symlin®);
G-protein coupled receptor 119 (GPR119) agonists (e.g., GSK-1292263, PSN-821, MBX-2982, APD-597, ARRY-981, ZYG-19, DS-8500, HM-47000, YH-Chem1);
GPR40 agonists (e.g., TUG-424, P-1736, P-11187, JTT-851, GW9508, CNX-011-67, AM-1638, AM-5262);
GPR120 agonists and GPR142 agonists;
systemic or low-absorbable TGR5 (GPBAR1=G-protein-coupled bile acid receptor 1) agonists (e.g., INT-777, XL-475, SB756050);
diabetes immunotherapeutics, for example: oral C-C chemokine receptor type 2 (CCR-2) antagonists (e.g., CCX-140, JNJ-41443532), interleukin 1 beta (IL-1B) antagonists (e.g., AC-201), or oral monoclonal antibodies (mAbs) (e.g., methalozamide, VVP808, PAZ-320, P-1736, PF-05175157, PF-04937319);
anti-inflammatory agents for the treatment of the metabolic syndrome and diabetes, for example: nuclear factor kappa B inhibitors (e.g., Triolex®);
adenosine monophosphate-activated protein kinase (AMPK) stimulants, for example: Imeglimin (PXL-008), Debio-0930 (MT-63-78), R-118;
inhibitors of 11-beta-hydroxysteroid dehydrogenase 1 (11-beta-HSD-1) (e.g., LY2523199, BMS770767, RG-4929, BMS816336, AZD-8329, HSD-016, BI-135585);
activators of glucokinase (e.g., PF-04991532, TTP-399 (GK1-399), GKM-001 (ADV-1002401), ARRY-403 (AMG-151), TAK-329, TMG-123, ZYGK1);
inhibitors of diacylglycerol O-acyltransferase (DGAT) (e.g., pradigastat (LCQ-908)), inhibitors of protein tyrosine phosphatase 1 (e.g., trodusquemine), inhibitors of glucose-6-phosphatase, inhibitors of fructose-1,6-bisphosphatase, inhibitors of glycogen phosphorylase, inhibitors of phosphoenol pyruvate carboxykinase, inhibitors of glycogen synthase kinase, inhibitors of pyruvate dehydrogenase kinase;
modulators of glucose transporter-4, somatostatin receptor 3 agonists (e.g., MK-4256);
one or more lipid lowering agents are also suitable as combination partners, for example: 3-hydroxy-3-methylglutaryl-coenzym-A-reductase (HMG-CoA-reductase) inhibitors such as simvastatin (e.g., Zocor®, Inegy®, Simcor®), atorvastatin (e.g., Sortis®, Caduet®), rosuvastatin (e.g., Crestor®), pravastatin (e.g., Lipostat®, Selipran®), fluvastatin (e.g., Lescol®), pitavastatin (e.g., Livazo®, Livalo®), lovastatin (e.g., Mevacor®, Advicor®), mevastatin (e.g., Compactin®), rivastatin, cerivastatin (Lipobay®), fibrates such as bezafibrate (e.g., Cedur® retard), ciprofibrate (e.g., Hyperlipen®), fenofibrate (e.g., Antara®, Lipofen®, Lipanthyl®), gemfibrozil (e.g., Lopid®, Gevilon®), etofibrate, simfibrate, ronifibrate, clinofibrate, clofibride, nicotinic acid and derivatives thereof (e.g., niacin, including slow release formulations of niacin), nicotinic acid receptor 1 agonists (e.g., GSK-256073), PPAR-delta agonists, acetyl-CoA-acetyltransferase (ACAT) inhibitors (e.g., avasimibe), cholesterol absorption inhibitors (e.g., ezetimibe, Ezetrol®, Zetia®, Liptruzet®, Vytorin®, S-556971), bile acid-binding substances (e.g., cholestyramine, colesevelam), ileal bile acid transport (IBAT) inhibitors (e.g., GSK-2330672, LUM-002), microsomal triglyceride transfer protein (MTP) inhibitors (e.g., lomitapide (AEGR-733), SLx-4090, granotapide), modulators of proprotein convertase subtilisin/kexin type 9 (PCSK9) (e.g., alirocumab (REGN727/SAR236553), AMG-145, LGT-209, PF-04950615, MPSK3169A, LY3015014, ALD-306, ALN-PCS, BMS-962476, SPC5001, ISIS-394814, 1B20, LGT-210, 1D05, BMS-PCSK9Rx-2, SX-PCK9, RG7652), LDL receptor up-regulators, for example liver selective thyroid hormone receptor beta agonists (e.g., eprotirome (KB-2115), MB07811, sobetirome (QRX-431), VIA-3196, ZYT1), HDL-raising compounds such as: cholesteryl ester transfer protein (CETP) inhibitors (e.g., anacetrapib (MK0859), dalcetrapib, evacetrapib, JTT-302, DRL-17822, TA-8995, R-1658, LY-2484595, DS-1442), or dual CETP/PCSK9 inhibitors (e.g., K-312), ATP-binding cassette (ABC1) regulators, lipid metabolism modulators (e.g., BMS-823778, TAP-301, DRL-21994, DRL-21995), phospholipase A2 (PLA2) inhibitors (e.g., darapladib, Tyrisa®, varespladib, rilapladib), ApoA-1 enhancers (e.g., RVX-208, CER-001, MDCO-216, CSL-112), cholesterol synthesis inhibitors (e.g., ETC-1002), lipid metabolism modulators (e.g., BMS-823778, TAP-301, DRL-21994, DRL-21995) and omega-3 fatty acids and derivatives thereof (e.g., icosapent ethyl (AMR101), Epanova®, AKR-063, NKPL-66, PRC-4016, CAT-2003);
bromocriptine (e.g., Cycloset©, Parlodel®), phentermine and phentermine formulations or combinations (e.g., Adipex-P, lonamin, Qsymia®), benzphetamine (e.g., Didrex®), diethylpropion (e.g., Tenuate®), phendimetrazin (e.g., Adipost®, Bontril®), bupropion and combinations (e.g., Zyban®, Wellbutrin XL®, Contrave®, Empatic©), sibutramine (e.g., Reductil®, Meridia®), topiramat (e.g., Topamax®), zonisamid (e.g., Zonegran®), tesofensine, opioid antagonists such as naltrexone (e.g., Naltrexin®, naltrexone+bupropion), cannabinoid receptor 1 (CB1) antagonists (e.g., TM-38837), melanin-concentrating hormone (MCH-1) antagonists (e.g., BMS-830216, ALB-127158(a)), MC4 receptor agonists and partial agonists (e.g., AZD-2820, RM-493), neuropeptide Y5 (NPY5) or NPY2 antagonists (e.g., velneperit, S-234462), NPY4 agonists (e.g., PP-1420), beta-3-adrenergic receptor agonists, leptin or leptin mimetics, agonists of the 5-hydroxytryptamine 2c (5HT2c) receptor (e.g., lorcaserin, Belviq®), pramlintide/metreleptin, lipase inhibitors such as cetilistat (e.g., Cametor®), orlistat (e.g., Xenical®, Calobalin®), angiogenesis inhibitors (e.g., ALS-L₁₀₂₃), betahistidin and histamine H3 antagonists (e.g., HPP-404), AgRP (agouti related protein) inhibitors (e.g., TTP-435), serotonin re-uptake inhibitors such as fluoxetine (e.g., Fluctine®), duloxetine (e.g., Cymbalta®), dual or triple monoamine uptake inhibitors (dopamine, norepinephrine and serotonin re-uptake) such as sertraline (e.g., Zoloft®), tesofensine, methionine aminopeptidase 2 (MetAP2) inhibitors (e.g., beloranib), and antisense oligonucleotides against production of fibroblast growth factor receptor 4 (FGFR4) (e.g., ISIS-FGFR4Rx) or prohibitin targeting peptide-1 (e.g., Adipotide®);
nitric oxide donors, AT1 antagonists or angiotensin II (AT2) receptor antagonists such as telmisartan (e.g., Kinzal®, Micardis®), candesartan (e.g., Atacand®, Blopress®), valsartan (e.g., Diovan®, Co-Diovan®), losartan (e.g., Cosaar®), eprosartan (e.g., Teveten®), irbesartan (e.g., Aprovel®, CoAprovel®), olmesartan (e.g., Votum®, Olmetec©), tasosartan, azilsartan (e.g., Edarbi®), dual angiotensin receptor blockers (dual ARBs), angiotensin converting enzyme (ACE) inhibitors, ACE-2 activators, renin inhibitors, prorenin inhibitors, endothelin converting enzyme (ECE) inhibitors, endothelin receptor (ET1/ETA) blockers, endothelin antagonists, diuretics, aldosterone antagonists, aldosterone synthase inhibitors, alpha-blockers, antagonists of the alpha-2 adrenergic receptor, beta-blockers, mixed alpha-/beta-blockers, calcium antagonists, calcium channel blockers (CCBs), nasal formulations of the calcium channel blocker diltiazem (e.g., CP-404), dual mineralocorticoid/CCBs, centrally acting antihypertensives, inhibitors of neutral endopeptidase, aminopeptidase-A inhibitors, vasopeptide inhibitors, dual vasopeptide inhibitors such as neprilysin-ACE inhibitors or neprilysin-ECE inhibitors, dual-acting AT receptor-neprilysin inhibitors, dual AT1/ETA antagonists, advanced glycation end-product (AGE) breakers, recombinant renalase, blood pressure vaccines such as anti-RAAS (renin-angiotensin-aldosteron-system) vaccines, AT1- or AT2-vaccines, drugs based on hypertension pharmacogenomics such as modulators of genetic polymorphisms with antihypertensive response, thrombocyte aggregation inhibitors, and others or combinations of any of these are suitable.

In certain exemplary embodiments, a “nucleic acid molecule” is according to the invention deoxyribonucleic acid (DNA) or ribonucleic acid (RNA). A nucleic acid molecule may according to the invention be in the form of a molecule which is single-stranded or double-stranded. A nucleic acid molecule according to the invention may be linear or covalently closed to form a circle.

The term “DNA”, as used herein, refers to a molecule which comprises deoxyribonucleotide residues and, in certain exemplary embodiments, is entirely or substantially composed of deoxyribonucleotide residues. “Deoxyribonucleotide”, as used herein, refers to a nucleotide, which lacks a hydroxyl group at the 2′-position of a beta-D-ribofuranosyl group. The term “DNA” includes isolated DNA, such as partially or completely purified DNA, essentially pure DNA, synthetic DNA, and recombinantly generated DNA. The term “DNA” also includes modified DNA, which differs from naturally occurring DNA by the addition, deletion, substitution and/or alteration of one or more nucleotides. Such alterations can include the addition of non-nucleotide material, such as to the end(s) of a DNA or internally, for example at one or more nucleotides of the DNA. Nucleotides in DNA molecules can also comprise non-standard nucleotides, such as non-naturally occurring nucleotides or chemically synthesized nucleotides.

Altered DNA molecules can be referred to as analogues or analogues of naturally-occurring DNA.

The term “RNA”, as used herein, refers to a molecule, which comprises ribonucleotide residues and is optionally entirely or substantially composed of ribonucleotide residues. “Ribonucleotide”, as used herein, refers to a nucleotide with a hydroxyl group at the 2′-position of a beta-D-ribofuranosyl group. The term “RNA” includes isolated RNA, such as partially or completely purified RNA, essentially pure RNA, synthetic RNA, and recombinantly generated RNA. The term “RNA” also includes modified RNA, which differs from naturally occurring RNA by the addition, deletion, substitution and/or alteration of one or more nucleotides. Such alterations can include the addition of a non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. Altered RNA molecules can be referred to as analogues or analogues of naturally-occurring RNA. According to the invention, “RNA” refers to single-stranded RNA or double stranded RNA. In one embodiment, the RNA is mRNA, e.g., In Vitro Transcribed RNA (IVT RNA) or synthetic RNA. The RNA may also be modified, e.g., with one or more modifications increasing the stability (e.g., the half-life) of the RNA. Such modifications are known to a person skilled in the art and include, for example, 5′-caps or 5′cap analogues.

When used in connection with nucleotides, the term “naturally occurring” refers to the bases adenine (A), cytosine (C), guanine (G), thymine (T), and uracil (U).

A nucleic acid molecule according to the present invention may be contained/comprised in a vector. The term “vector”, as used herein, includes all vectors known to the skilled person, including plasmid vectors, cosmid vectors, phage vectors (e.g., lambda phage), viral vectors (e.g., adenoviral or baculoviral vectors), or artificial chromosome vectors (e.g., Bacterial Artificial Chromosomes (BACs), Yeast Artificial Chromosomes (YACs), or P1 Artificial Chromosomes (PACs)). Said vectors include expression as well as cloning vectors. Expression vectors include plasmids as well as viral vectors and generally contain a desired coding sequence and appropriate DNA sequences necessary for the expression of an operably linked coding sequence in a particular host organism (e.g., a bacterium, a yeast, a plant, an insect, a mammal or the like) or in an in vitro expression system. Cloning vectors are generally used to engineer and amplify a certain desired DNA fragment and may lack functional sequences needed for expression of the desired DNA fragments.

Alternatively, a nucleic acid molecule according to the present invention may be integrated into a genome, e.g., the genome of a host cell. Means and methods to integrate a particular nucleic acid molecule into a genome are well-known to a person skilled in the art.

The term “cell” or “host cell”, as used herein, refers to an intact cell, i.e., a cell with an intact membrane that has not released its normal intracellular components such as enzymes, organelles, or genetic material. In certain exemplary embodiments, an intact cell is a viable cell, i.e., a living cell capable of carrying out its normal metabolic functions. In certain exemplary embodiments, a cell or a host cell is any cell which can be transfected or transformed with an exogenous nucleic acid. In certain exemplary embodiments, the cell, when transfected or transformed with an exogenous nucleic acid and transferred to a recipient, can express the nucleic acid in the recipient.

The term “cell” includes prokaryotic cells, such as bacterial cells, and eukaryotic cells, such as yeast cells, fungal cells or mammalian cells. Suitable bacterial cells include, but are not limited to, cells from gram-negative bacterial strains, such as strains of Escherichia coli, Proteus, and Pseudomonas, and gram-positive bacterial strains, such as strains of Bacillus, Streptomyces, Staphylococcus, and Lactococcus. Suitable fungal cells include, but are not limited to, cells from the species of Trichoderma, Neurospora, and Aspergillus. Suitable yeast cells include, but are not limited to, cells from the species of Saccharomyces (for example, Saccharomyces cerevisiae), Schizosaccharomyces (for example, Schizosaccharomyces pombe), Pichia (for example, Pichia pastoris and Pichia methanolica), and Hansenula. Suitable mammalian cells include, but are not limited to, for example, CHO cells, BHK cells, HeLa cells, COS cells, HEK-293 and the like. In one embodiment, HEK-293 cells are used. However, amphibian cells, insect cells, plant cells, and any other cells used in the art for the expression of heterologous proteins can be used, as well. In certain exemplary embodiments, mammalian cells (e.g., cells from humans, mice, hamsters, pigs, goats, or primates) are used for adoptive transfer.

Suitable cells may be derived from a large number of tissue types and include primary cells and cell lines, such as cells of the immune system (e.g., antigen-presenting cells such as dendritic cells and T cells, stem cells such as hematopoietic stem cells and mesenchymal stem cells) as well as any other cell types.

An “antigen-presenting cell”, as used herein, is a cell that displays antigen in the context of major histocompatibility complex on its surface. A T cell may recognize a histocompatibility complex using its T cell receptor (TCR).

A cell or host cell may be isolated or part of a tissue or organism, in particular a “non-human organism”. The term “non-human organism”, as used herein, is meant to include non-human primates or other animals, e.g., mammals, such as cows, horses, pigs, sheep, goats, dogs, cats, rabbits or rodents (e.g., mice, rats, guinea pigs and hamsters).

A pharmaceutical composition in accordance with the present invention comprises one or more carriers and/or excipients, all of which are pharmaceutically acceptable. The term “pharmaceutically acceptable”, as used herein, refers to the non-toxicity of a material which, in certain exemplary embodiments, does not interact with the action of the active agent of the pharmaceutical composition.

The term “carrier”, as used herein, refers to an organic or inorganic component, of a natural or synthetic nature, in which the active component is combined in order to facilitate, enhance or enable application. According to the invention, the term “carrier” also includes one or more compatible solid or liquid fillers, diluents or encapsulating substances, which are suitable for administration to a subject.

Suitable carrier substances for parenteral administration include, but are not limited to, sterile water, Ringer's solution, Lactated Ringer's solution, physiological saline, bacteriostatic saline (e.g., saline containing 0.9% benzyl alcohol), phosphate-buffered saline (PBS), Hank's solution, polyalkylene glycols, hydrogenated naphthalenes and, in particular, biocompatible lactide polymers, lactide/glycolide copolymers or polyoxyethylene/polyoxy-propylene copolymers.

The term “excipient”, as used herein, is intended to include all substances which may be present in a pharmaceutical composition and which are not active ingredients, such as salts, binders (e.g., lactose, dextrose, sucrose, trehalose, sorbitol, mannitol), fillers, lubricants, thickeners, surface active agents, preservatives, emulsifiers, buffer substances, flavoring agents, or colorants.

Salts that are not pharmaceutically acceptable may be used for preparing pharmaceutically acceptable salts and are included in the invention. Pharmaceutically acceptable salts of this kind comprise, in a non-limiting way, those prepared from the following acids: hydrochloric, hydrobromic, sulfuric, nitric, phosphoric, maleic, acetic, salicylic, citric, formic, malonic, succinic, and the like. Pharmaceutically acceptable salts may also be prepared as alkali metal salts or alkaline earth metal salts, such as sodium salts, potassium salts or calcium salts. Salts may be added to adjust the ionic strength or tonicity of a pharmaceutical composition.

Suitable preservatives for use in a pharmaceutical composition include, but are not limited to, antioxidants, citric acid, sodium citrate, benzalkonium chloride, chlorobutanol, cysteine, methionine, parabens, thimerosal, phenol, cresol, and mixtures thereof.

Suitable buffer substances for use in a pharmaceutical composition include, but are not limited to, acetic acid in a salt, citric acid in a salt, boric acid in a salt, phosphoric acid in a salt, and tris(hydroxymethyl)aminomethane (Tris, THAM, trometamol).

In certain exemplary embodiments, a pharmaceutical composition in accordance with the present invention is sterile. Pharmaceutical compositions may be provided in a uniform dosage form and may be prepared in any manner known by those of skill in the art. A pharmaceutical composition may, e.g., be in the form of a solution or suspension.

A pharmaceutical composition may also be formulated as a stable lyophilized product that is reconstituted with an appropriate diluent, which, optionally, comprises one or more excipients as defined above.

A pharmaceutical composition in accordance with the present invention may further comprise at least one other active pharmaceutical ingredient in addition to a GLP-1R agonistic peptide.

As used herein, the term “kit of parts (in short: kit)” refers to an article of manufacture comprising one or more containers and, optionally, a data carrier. Said one or more containers may be filled with one or more of the above mentioned agents of the present invention, e.g., GLP-1R agonistic peptides, fusion proteins, pharmaceutical compositions and related agents, such as nucleic acid molecules and host cells. Additional containers may be included in the kit that contain, e.g., diluents, buffers and further reagents. Said data carrier may be a non-electronical data carrier, e.g., a graphical data carrier such as an information leaflet, an information sheet, a bar code or an access code, or an electronical data carrier such as a compact disk (CD), a digital versatile disk (DVD), a microchip or another semiconductor-based electronical data carrier. The access code may allow the access to a database, e.g., an internet database, a centralized, or a decentralized database. Said data carrier may comprise instructions for the use of the agents of the present invention, e.g., GLP-1R agonistic peptides, fusion molecules pharmaceutical compositions and related agents, such as nucleic acid molecules and host cells, as described herein.

The agents and compositions described herein may be administered via any conventional route, e.g., orally, pulmonary administration, by inhalation or parenterally, including by injection or infusion. In one embodiment, parenteral administration is used, e.g., intravenously, intraarterially, subcutaneously, intradermally or intramuscularly. The agents and compositions described herein may also be administered through sustained release administration.

Pharmaceutical compositions suitable for parenteral administration typically comprise a sterile aqueous or non-aqueous preparation of the active compound, which is optionally isotonic to the blood of the recipient. Examples of compatible carriers/solvents/diluents are sterile water, Ringer's solution, Lactated Ringer's solution, physiological saline, bacteriostatic saline (e.g., saline containing 0.9% benzyl alcohol), PBS and Hank's solution. In addition, usually sterile, fixed oils may be used as solution or suspension medium.

The agents and compositions described herein are usually administered in therapeutically effective amounts. A “therapeutically effective amount” refers to the amount, which achieves a desired therapeutic reaction or a desired therapeutic effect alone or together with further doses, optionally without causing or only minimally causing unacceptable or unwanted side-effects.

In certain exemplary embodiments, the case of treatment of a particular disease, a particular disorder, or a particular condition, the desired reaction can relate to inhibition of the course of the disease, disorder or condition. This comprises slowing down the progress of the disease, disorder or condition and, in particular, interrupting or reversing the progress of the disease, disorder or condition. The desired reaction in a treatment of a disease, disorder or condition may also be delay of the onset or a prevention of the onset of said disease, disorder or condition. An effective amount of an agent or composition described herein will depend on the condition to be treated, the severeness of the disease, disorder or condition, the individual parameters of the subject, including age, physiological condition, size and weight, the duration of treatment, the type of an accompanying therapy (if present), the specific route of administration and similar factors. Accordingly, the doses administered of the agents described herein may depend on various of such parameters. In the case that a reaction in a subject is insufficient with an initial dose, higher doses (or effectively higher doses achieved by a different, more localized route of administration) may be used.

According to the invention, the term “disease, disorder or condition” refers to any pathological or unhealthy state, in particular obesity, being overweight, metabolic syndrome, diabetes mellitus, diabetic retinopathy, hyperglycemia, dyslipidemia, NASH and/or atherosclerosis.

The term “obesity”, as used herein, refers to a medical condition in which excess body fat has accumulated to the extent that it may have a negative effect on health. In terms of a human (adult) subject, obesity can be defined as a body mass index (BMI) greater than or equal to 30 kg/m² (BMI≥30 kg/m²).

The phrase “being overweight”, as used herein, refers to a medical condition in which the amount of body fat is higher than is optimally healthy. In terms of a human (adult) subject, obesity can be defined as a body mass index (BMI) greater than or equal to 25 kg/m² (e.g., 25 kg/m²≤BMI<30 kg/m²).

The BMI is a simple index of weight-for-height that is commonly used to classify overweight and obesity in adults. It is defined as a person's weight in kilograms divided by the square of his/her height in meters (kg/m²).

“Metabolic syndrome”, as used herein, refers to a clustering of at least three of the following medical conditions: abdominal (central) obesity (e.g., defined as waist circumference≥94 cm for Europid men and ≥80 cm for Europid women, with ethnicity specific values for other groups), elevated blood pressure (e.g., 130/85 mmHg or higher), elevated fasting plasma glucose (e.g., at least 100 mg/dL), high serum triglycerides (e.g., at least 150 mg/dL), and low high-density lipoprotein (HDL) levels (e.g., less than 40 mg/dL for males and less than 50 mg/dL for females).

“Diabetes mellitus” (also simply referred to as “diabetes”), as used herein, refers to a group of metabolic diseases characterized by high levels of blood glucose resulting from defects in insulin production, insulin action, or both. In one embodiment, diabetes mellitus is selected from the group consisting of type 1 diabetes mellitus, type 2 diabetes mellitus, gestational diabetes mellitus, Late onset Autoimmune Diabetes in the Adult (LADA), Maturity Onset Diabetes of the Young (MODY) and other types of diabetes resulting from specific genetic conditions, drugs, malnutrition, infections and other illnesses. The current WHO diagnostic criteria for diabetes mellitus are as follows: fasting plasma glucose≥7.0 mmol/I (126 mg/dL) or 2-hour plasma glucose≥11.1 mmol/I (200 mg/dL).

“Type 1 diabetes mellitus” (also known as “Insulin-Dependent Diabetes Mellitus (IDDM)” or “juvenile diabetes”), as used herein, is a condition characterized by high blood glucose levels caused by total lack of insulin. This occurs when the body's immune system attacks the insulin producing beta cells in the pancreas and destroys them. The pancreas then produces little or no insulin. Pancreatic removal or disease may also lead to loss of insulin-producing beta cells. Type 1 diabetes mellitus accounts for between 5% and 10% of cases of diabetes.

“Type 2 diabetes mellitus” (also known as “Non-Insulin-Dependent Diabetes (NIDDM)” or “adult-onset diabetes”), as used herein, is a condition characterized by excess glucose production in spite of the availability of insulin, and circulating glucose levels remain excessively high as a result of inadequate glucose clearance (insulin action). Type 2 diabetes mellitus may account for about 90% to 95% of all diagnosed cases of diabetes.

“Gestational diabetes”, as used herein, is a condition in which women without previously diagnosed diabetes exhibit high blood glucose levels during pregnancy (especially during the third trimester). Gestational diabetes affects 3-10% of pregnancies, depending on the population studied.

“Late onset Autoimmune Diabetes in the Adult (LADA)” (also referred to as “slow onset type 1 diabetes”), as used herein, is a form of type 1 diabetes mellitus that occurs in adults, often with a slower course of onset.

“Maturity Onset Diabetes of the Young (MODY)”, as used herein, refers to a hereditary form of diabetes caused by mutations in an autosomal dominant gene disrupting insulin production.

“Diabetic retinopathy”, as used herein, is an ocular disease induced by the metabolic disarrangements occurring in diabetic patients and leads to progressive loss of vision.

The term “hyperglycemia”, as used herein, refers to an excess of sugar (glucose) in the blood.

The term “dyslipidemia”, as used herein, refers to a disorder of lipoprotein metabolism, including lipoprotein overproduction (“hyperlipidemia”) or deficiency (“hypolipidemia”). Dyslipidemias may be manifested by elevation of the total cholesterol, low-density lipoprotein (LDL) cholesterol and/or triglyceride concentrations, and/or a decrease in high-density lipoprotein (HDL) cholesterol concentration in the blood.

Non-Alcoholic SteatoHepatitis (NASH), as used herein, refers to a liver disease characterized by an accumulation of fat (lipid droplets), along with inflammation and degeneration of hepatocytes. Once initiated, the disease is accompanied with a high risk of cirrhosis, a state wherein liver functions are altered that can progress to liver insufficiency. Thereafter, NASH often progresses to liver cancer.

“Atherosclerosis”, as used herein, refers to a vascular disease characterized by irregularly distributed lipid deposits called plaque in the intima of large and medium-sized arteries that may cause narrowing of arterial lumens and proceed to fibrosis and calcification. Lesions are usually focal and progress slowly and intermittently. Occasionally, plaque rupture occurs leading to obstruction of blood flow resulting in tissue death distal to the obstruction. Limitation of blood flow accounts for most clinical manifestations, which vary with the distribution and severity of the obstruction.

The term “medicament”, as used herein, refers to a substance and/or composition used in therapy, i.e., in the treatment of a disease or disorder.

The term “treat”, as used herein, refers to the administration of a compound or composition or a combination of compounds or compositions to a subject in order to: prevent, ameliorate, or eliminate a disease, disorder or condition in a subject; arrest or slow disease, disorder or condition in a subject; inhibit or slow the development of a new disease, disorder or condition in a subject; decrease the frequency or severity of symptoms and/or recurrences in a subject who currently has or who previously has had a disease, disorder or condition; and/or prolong, i.e., increase, the lifespan of a subject.

In particular, the phrases “treating a disease, disorder or condition” and “treatment of a disease, disorder or condition” include curing, shortening the duration of, ameliorating, preventing, slowing down or inhibiting progression or worsening, or preventing or delaying the onset of a disease, disorder or condition or the symptoms thereof.

The term “subject”, according to the invention, refers to a subject for treatment, in particular a diseased subject (also referred to as “patient”), including, but not limited to, a human being, a non-human primates, or other animals, e.g., mammals, such as cows, horses, pigs, sheep, goats, dogs, cats, rabbits or rodents (e.g., mice, rats, guinea pigs or hamsters). In one embodiment, the subject/patient is a human being.

The present invention is now further described by reference to the following Examples, which are intended to illustrate, and not to limit, the scope of the present invention.

### EXAMPLES

### Example 1: Determining the Optimal GLP-1RA/FGF21 Activity Ratio by Systems Pharmacology Modelling

Improved mechanistic insights into pharmacological effects of GLP-1RA/FGF21 fusion proteins in humans were used to identify optimal GLP-1RA/FGF21 potency ratio. A mechanistic systems pharmacology model was developed describing effects of GLP-1 and FGF21 on glucose, lipid, and energy metabolism in humans (Cuevas-Ramos et al. (2009) Curr Diabetes Rev 5(4): 216-220; Deacon et al. (2011) Rev Diabet Stud 8(3): 293-306; Kim et al. (2008) Pharmacol Rev 60(4): 470-512; Kharitonenkov et al. (2014) Mol Metab 3(3): 221-229).

The model represented relevant pathways for GLP-1 and FGF21 effects. Glycemic control (i.e., HbA1c, fasting plasma glucose, postprandial glucose), lipid parameters (i.e., plasma triglycerides, fatty acids, cholesterol), and energy balance (i.e., body weight, food intake, energy expenditure) were captured to assess therapeutic response to simulated drug treatment (e.g., GLP-1RA/FGF21 fusion protein, Liraglutide, FGF21 analog LY2405319). For LY2405319, see Kharitonenkov et al. (2013) PLoS ONE 8(3): e58575.

The model covered key aspects of glucose homeostasis controlled by the hormones insulin, glucagon, and certain incretins (e.g., GLP-1, GIP). The major model endpoint regarding glycemic control was HbA1c. HbA1c is a common clinical endpoint used to estimate average plasma glucose concentrations over the previous several months. HbA1c was estimated within the model using the linear correlation between mean plasma glucose and HbA1c as reported by Nathan et al. (2008) Diabetes Care 31(8): 1473-1478.

The model incorporated triglyceride and fatty acid metabolism at a level appropriate to handle basic lipid metabolism, including the representation of cholesterol. HDL and non-HDL, i.e., LDL plus VLDL cholesterol, are the circulating lipoproteins. The representation of lipid metabolism allowed simulating the impact of FGF21 compounds on lipids and the interaction with statins.

FGF21 compounds had significant effects on lipid concentrations (Gaich et al. (2013) Cell Metab 18(3): 333-340; Fisher et al. (2011) Endocrinology 152(8): 2996-3004).

Weight loss or gain in the model was measured as changes in body adipose mass. There was a direct relationship between fat mass and body weight (Broyles et al. (2011) Br J Nutr 105(8): 1272-1276). Food intake was based on basal and resting metabolic rate (Amirkalali et al. (2008) Indian J Med Sci 62(7): 283-290). Body adipose mass stayed constant when energy expenditure equaled caloric intake. Therapy effects on food intake were implemented in the model using the formulation of Gobel et al. (2014) (Obesity (Silver Spring) 22(10): 2105-2108).

Food was considered to be carbohydrate (glucose equivalents), fat (fatty acid equivalents), and protein (amino acid equivalents). All nutrients entered the stomach, passed through a delay node and then entered a three-compartment gastrointestinal tract. The gastrointestinal tract design was based on work done by Bastianelli et al. (1996) (J Anim Sci 74(8): 1873-1887) and Worthington (1997) (Med Inform (Lond) 22(1): 35-45) regarding food digestion and absorption.

Nutrients, hormones, drugs, and disease conditions can cause delays in gastric emptying. Under healthy conditions, the gastric emptying rate depends on the size of the meal, its energy density, and the amount of nutrients in the stomach (Achour et al. (2001) Eur J Clin Nutr 55(9): 769-772; Fouillet et al. (2009) Am J Physiol Regul Integr Comp Physiol 297(6): R1691-1705). Individuals with diabetes often have a delay in glucose absorption observed with an oral glucose tolerance test or a meal test (Bharucha et al. (2009) Clin Endocrinol (Oxf) 70(3): 415-420; Chang et al. (2012) Diabetes Care 35(12): 2594-2596). This delay is attributed to a slowing of gastric emptying. A delay in travel between the stomach and small intestine was added in the model of this example to account for delayed gastric emptying in diabetic subjects. Drugs and hormones (e.g., GLP-1) can affect the vagal tone of the stomach, which reduces mechanical mixing and/or peristalsis, and also slows gastric emptying (Jelsing et al. (2012) Diabetes Obes Metab 14(6): 531-538; Little et al. (2006) J Clin Endocrinol Metab 91(5): 1916-1923; Nauck et al. (2011) Diabetes 60(5): 1561-1565; van Can et al. (2013) Int J Obes (Lond) 38(6): 784-93).

One aim of this investigation was preventing GLP-1 related adverse effects, i.e., nausea and vomiting (Lean et al. (2014) Int J Obes (Lond) 38(5): 689-697). Gastric emptying measures provided an estimate of adverse events such as nausea and vomiting that correlated with low rates of gastric emptying. Hence, a marker for gastric adverse events in the model was the sum of gastric emptying rate.

Different virtual patients were implemented in a model platform representing healthy and type 2 diabetic patients at different stages of the disease. Moreover, the virtual patients covered different degrees of obesity and dyslipidemia. The virtual patients represented variability in disease severity and pathophysiology and phenotypic variability observed in the clinic.

Several therapies were implemented in the model, i.e., GLP-1RA/FGF21 fusion protein, Liraglutide, FGF21 analog LY2405319, Metformin, Atorvastatin, Sitagliptin, and human insulin. These therapies could be switched on or off in the simulations. The virtual patient was assumed to be on a background of Metformin and Atorvastatin when administered the GLP-1RA/FGF21 fusion protein.

Virtual GLP-1RA/FGF21 fusion proteins were implemented in the model described in this example. The fusion protein contained both FGF21 and GLP-1 agonistic activities, and it had the same effects as both FGF21 and GLP-1 receptor agonists. The pharmacokinetic profiles of the virtual fusion proteins were assumed to be similar to Dulaglutide (Geiser et al. (2016) Clin Pharmacokinet 55(5): 625-34).

The model was validated by comparison with numerous data sets. The simulation results were qualitatively consistent with relevant data and knowledge, e.g., Hellerstein et al. (1997) J Clin Invest 100(5): 1305-1319; Muscelli et al. (2008) Diabetes 57(5): 1340-1348. The model matched relevant quantitative test data, e.g., Aschner et al. (2006) Diabetes Care 29(12): 2632-2637; Dalla Man, Caumo et al. (2005) Am J Physiol Endocrinol Metab 289(5): E909-914; Dalla Man et al. (2005) Diabetes 54(11): 3265-3273; Fiallo-Scharer (2005) J Clin Endocrinol Metab 90(6): 3387-3391; Hahn et al. (2011) Theor Biol Med Model 8: 12; Herman et al. (2005) Clin Pharmacol Ther 78(6): 675-688; Herman et al. (2006) J Clin Pharmacol 46(8): 876-886 and J Clin Endocrinol Metab 91(11): 4612-4619; Hojlund et al. (2001) Am J Physiol Endocrinol Metab 280(1): E50-58; Monauni et al. (2000) Diabetes 49(6): 926-935; Nauck et al. (2009) Diabetes Care 32(1): 84-90; Nauck et al. (1993) J Clin Invest 91(1): 301-307; Nauck et al. (2004) Regul Pept 122(3): 209-217; Tzamaloukas et al. (1989) West J Med 150(4): 415-419; Sikaris (2009) J Diabetes Sci Technol 3(3): 429-438; Vicini and Cobelli (2001) Am J Physiol Endocrinol Metab 280(1): E179-186; Vollmer et al. (2008) Diabetes 57(3): 678-687.

Existing therapies were implemented in the model for direct comparison, including FGF21 analog and a GLP-1 receptor agonist. The FGF21 analog's effects were validated with clinical data, e.g., Gaich et al. (2013) Cell Metab 18(3): 333-340. The GLP-1 receptor agonist Liraglutide was a direct competitor for the target, and its implementation was compared with various clinical data, e.g., data described in Jacobsen et al. (2009) Br J Clin Pharmacol 68(6): 898-905; Elbrond et al. (2002) Diabetes Care 25(8): 1398-1404; Chang et al. (2003) Diabetes 52(7): 1786-1791; Kolterman et al. (2003) J Clin Endocrinol Metab 88(7): 3082-3089; Degn et al. (2004) Diabetes 53(5): 1187-1194; Kolterman et al. (2005) Am J Health Syst Pharm 62(2): 173-181; Vilsboll et al. (2008) Diabet Med 25(2): 152-156; Buse et al. (2009) Lancet 374(9683): 39-47; Jelsing et al. (2012) Diabetes Obes Metab 14(6): 531-538; Hermansen et al. (2013) Diabetes Obes Metab 15(11): 1040-1048; Suzuki et al. (2013) Intern Med 52(10): 1029-1034; van Can et al. (2013) Int J Obes (Lond) 38(6): 784-93); Zinman et al. (2009) Diabetes Care 32(7): 1224-1230; Russell-Jones et al. (2009) Diabetologia 52(10): 2046-2055; Pratley et al. (2011) Int J Clin Pract 65(4): 397-407; Nauck et al. (2013) Diabetes Obes Metab 15(3): 204-212; Flint et al. (2011) Adv Ther 28(3): 213-226; Kapitza et al. (2011) Adv Ther 28(8): 650-660; and Astrup et al. (2012) Int J Obes (Lond) 36(6): 843-854.

The model platform allowed simulation of beneficial and adverse effects of virtual GLP-1 RA/FGF21 fusion proteins with varying activity ratios. Effective FGF21-mediated EC50 values were set constant to those derived from Gaich et al. (2013) Cell Metab 18(3): 333-340. Effective GLP-1-mediated EC50 values were reduced by a factor of 2 to 600 in increments of 1 relative to endogenous GLP-1 (Table 1).

TABLE 1
GLP-1R agonist/FGF21 fusion protein pharmacodynamics (EC50 values).

| | | | | | Effective |
|---|---|---|---|---|---|
| | Effective GLP-1-Mediated EC50 Values | | | | FGF21- |
| | Peripheral | | | | Mediated |
| Potency | Glucose | Insulin | Gastric | Food | EC50 |
| Ratio* | Uptake | Release | Emptying | Intake | Values** |
| 1 | 35 pM | 20 pM | 50 pM | 80 pM | 3547 pM |
| 100 | 3500 pM | 2000 pM | 5000 pM | 8000 pM | 3547 pM |

*Relative to endogenous GLP-1
**FGF21 EC50 values were set assuming half maximal effect per Gaich et al. (2013) Cell Metab 18(3): 333-340

For each virtual fusion protein, the exposure-response relation was simulated for relevant pharmacodynamic endpoints, i.e., HbA1c, triglycerides, fatty acids, non-HDL cholesterol, and adipose mass. As a marker for GLP-1-mediated adverse events, gastric emptying rate was used. 52 weeks treatment of an average obese dyslipidemic type 2 diabetic virtual patient with GLP-1RA/FGF21 fusion proteins was simulated for a broad dose range. After treatment for 52 weeks, all relevant pharmacodynamic endpoints were expected to reach steady state. For each endpoint the half maximal effective concentration (EC50 value) was determined from the exposure-response curves. The EC50 values varied with the activity ratio, especially for the mainly GLP-1-mediated endpoints HbA1c and gastric emptying rate. FIG. 1 depicts the EC50 values depending on the GLP-1 attenuation factor. An increased GLP-1 attenuation factor indicated a reduction in GLP-1R agonistic activity.

This procedure allowed the identification of relevant activity ratios, for which adverse effects were observed at higher plasma levels as compared to the plasma levels that mediated pharmacodynamics effects. For GLP-1 attenuation factors greater than 9, the EC50 of GLP-1-mediated gastrointestinal adverse effects was greater than the EC50 of pharmacodynamic effects. Hence, gastric adverse effects occurred at higher plasma levels than levels needed to achieve pharmacodynamic effects. It was possible to elucidate doses that provided all desirable pharmacodynamic effects while avoiding GLP-1-mediated gastrointestinal adverse effects.

The maximal EC50 value for gastric emptying rate was reached at attenuation factor 531. The maximal distance between adverse and mean pharmacodynamics effects was reached at attenuation factor 482 (FIG. 2). Therefore, activity ratios beyond 1:482 were not relevant. The maximal distance between maximum of pharmacodynamics (HbA1c) and adverse effects was 319. The maximal distance between maximum of pharmacodynamics (HbA1c) and adverse effects normalized by spreading of FGF21- (lipids) and GLP-1-mediated effects (HbA1c) was 121.

GLP-1RA/FGF21 fusion proteins with potency ratios between 1:10 and 1:482 were predicted to be most beneficial in improving lipid profile, body weight, and glucose metabolism and likely caused no significant adverse events based on gastric emptying response. Lower potency ratios were likely not good candidates based on their predicted strong inhibition of gastric emptying and potential for adverse events. Higher potency ratios were thought likely to be not sufficiently effective and therefore not competitive.

12 weeks of treatment of an average obese dyslipidemic type 2 diabetic virtual patient with GLP-1RA/FGF21 fusion proteins was simulated for a broad dose range, since the primarily GLP-1-mediated parameter of HbA1c levels clinically reaches steady state after 12 weeks of treatment with GLP-1 receptor agonists and FGF21 agents known in the art.

FIG. 3 depicts the EC50 values obtained versus GLP-1 attenuation factor over a 12 weeks simulation period. For GLP-1 attenuation factors greater than 18, the EC50 of GLP-1-mediated gastrointestinal adverse effect was greater than EC50 of pharmacodynamic effects. The maximal EC50 value for gastric emptying rate was reached at attenuation factor 501. The maximal distance between adverse and mean pharmacodynamics effects was reached at attenuation factor 469 (FIG. 4). The maximal distance between maximum of pharmacodynamics (HbA1c) and adverse effects was 313. The maximal distance between maximum of pharmacodynamics (HbA1c) and adverse effects normalized by spreading of FGF21- (lipids) and GLP-1-mediated effects (HbA1c) was 123.

Efficacy and potential for adverse events for GLP-1 RA/FGF21 fusion proteins with different activity ratios were investigated by means of the described systems pharmacology approach. Fusion proteins with presumably calculated ideal potency ratios were identified, predicted to be beneficial in improving lipid profile, body weight, and glycemic control while likely not causing significant adverse GLP-1 RA associated effects based on gastric emptying response. Compounds with the selected model-informed potency ratios were predicted to provide a good efficacy versus risk profile.

### Example 2: Expression of Homodimeric GLP-1RA/FGF21 Fusion Proteins in HEK-293, CHO and E. coli Cells and Chemical Synthesis of Isolated GLP-1R Agonistic Peptides

GLP-1RA/FGF21 Fc fusion proteins were produced by transient transfection in HEK-293 or CHO cells. DNA sequences of the fusion proteins were N-terminally fused to an IL2 signal sequence (SEQ ID NO: 246) followed by a histidine-rich sequence (His-tag) and a TEV protease-cleavage site (SEQ ID NO: 247 or 248). The signal sequence was required for secretion of the desired proteins into the culture medium. The proteins were purified from the culture supernatant using immobilized metal-ion affinity chromatography (IMAC) (cOmplete His-Tag Purification Column™, Roche). After elution from the IMAC-column, the N-terminal His-tag was optionally cleaved by the addition of Tobacco Etch Virus (TEV) protease. After His-tag cleavage, the cleavage reaction solution was passed a second time over an IMAC-column (cOmplete His-Tag Purification Column™, Roche), collecting the (His-tag-free) flow-through fraction. The protein was further purified using Protein A affinity chromatography (rProtein A Sepharose, GE Healthcare) and a gel filtration column with phosphate buffered saline (PBS, Gibco) as running buffer. Fractions containing the desired proteins were collected, pooled, concentrated and stored at −80° C. until further usage.

The FGF21 protein of SEQ ID NO: 252 (mature human wild-type FGF21 with an additional N-terminal Gly; referred to as G-FGF21 herein) was expressed in E. coli. The DNA sequence of the FGF21 protein was N-terminally fused to a Histidine-rich sequence (His-tag) and a TEV or SUMO protease-cleavage site (SEQ ID NO: 248 or 249). The desired protein was purified using immobilized metal-ion affinity chromatography (IMAC) (HisTrap HP, GE Healthcare) followed by cleavage of the N-terminal His-tag by addition of TEV or SUMO protease. After His-tag cleavage, the cleavage reaction solution was purified using an ion exchange column (Source 15, GE Healthcare), followed by a gel filtration column (Superdex 75, GE Healthcare) using phosphate buffered saline (PBS, Gibco) as running buffer. Fractions containing the desired protein were collected, pooled, concentrated and stored at −80° C. until further usage.

In an alternative approach, fusion proteins were produced by expression in E. coli inclusion bodies followed by a refolding step in which folded fusion protein was obtained by unfolding the inclusion bodies in Tris-buffered guanidinium chloride solution and refolding by dilution in buffer without chaotrophic salt. Fusion proteins were purified using Protein A affinity chromatography (MabSelect SuRe, GE Healthcare) followed by cleavage of the N-terminal pre-sequence by addition of TEV protease. The cleavage reaction solution was purified using an anion exchange column (POROS 50 HQ, ThermoFisher). Fractions containing the desired proteins were collected and pooled. Final buffer conditions and protein concentration were established by an ultrafiltration/diafiltration step using PBS (Gibco). Samples were stored at −80° C. until further usage.

Whereas fusion proteins were produced by recombinant methods (see above), isolated peptidic GLP-1R agonists were chemically synthesized.

More particularly, peptides were synthesized using the following manual synthesis procedure:

0.3 g Desiccated Rink amide MBHA Resin (0.66 mmol/g) was placed in a polyethylene vessel equipped with a polypropylene filter. Resin was swollen in DCM (15 ml) for 1 hour and DMF (15 mL) for one hour. The Fmoc group on the resin was de-protected by treating it twice with 20% (v/v) piperidine/DMF solution for 5 and 15 minutes. The resin was washed with DMF/DCM/DMF (6:6:6 times each). A Kaiser test (quantitative method) was used for the conformation of removal of Fmoc from solid support. The C-terminal Fmoc-amino acid (5 equivalent excess corresponding to resin loading) in dry DMF was added to the de-protected resin and coupling of the next Fmoc-amino acid was initiated with 5 equivalent excess of DIC and HOBT in DMF. The concentration of each reactant in the reaction mixture was approximately 0.4 M. The mixture was rotated on a rotor at room temperature for 2 hours. Resin was filtered and washed with DMF/DCM/DMF (6:6:6 times each). Kaiser tests performed on peptide resin aliquots upon completion of coupling was negative (no color on the resin). After the first amino acid attachment, the unreacted amino group, if any, in the resin was capped used acetic anhydride/pyridine/DCM (1:8:8) for 20 minutes to avoid any deletion of the sequence. After capping, resin was washed with DCM/DMF/DCM/DMF (6/6/6/6 time each). The Fmoc group on the C-terminal amino acid attached peptidyl resin was deprotected by treating it twice with 20% (v/v) piperidine/DMF solution for 5 and 15 minutes. The resin was washed with DMF/DCM/DMF (6:6:6 times each). The Kaiser tests performed on peptide resin aliquots upon completion of Fmoc-deprotection were positive.

The remaining amino acids in target sequence on Rink amide MBHA Resin were sequentially coupled using Fmoc AA/DIC/HOBt method using 5 equivalent excess corresponding to resin loading in DMF. The concentration of each reactant in the reaction mixture was approximately 0.4 M. The mixture was rotated on a rotor at room temperature for 2 hours. Resin was filtered and washed with DMF/DCM/DMF (6:6:6 times each). After each coupling step and Fmoc deprotection step, a Kaiser test was carried out to confirm the completeness of the reaction.

After the completion of the linear sequence, the ε-amino group of lysine was used as a branching point or a modification point and was deprotected by using 2.5% hydrazine hydrate in DMF for 15 minutes two times and washed with DMF/DCM/DMF (6:6:6 time each). The γ-carboxyl end of glutamic acid was attached to the ε-amino group of Lys using Fmoc-Glu(OH)-OtBu with DIC/HOBt method (using 5 equivalent excess with respect to resin loading) in DMF. The mixture was rotated on a rotor at room temperature for 2 hours. The resin was filtered and washed with DMF/DCM/DMF (6×30 mL each). The Fmoc group on the glutamic acid was de-protected by treating it twice with 20% (v/v) piperidine/DMF solution for 5 and 15 minutes (25 mL each). The resin was washed with DMF/DCM/DMF (6:6:6 times each). A Kaiser test on peptide resin aliquot upon completion of Fmoc-deprotection was positive.

If the side-chain branching also contained one more γ-glutamic acid, a second Fmoc-Glu(OH)-OtBu was used for the attachment to the free amino group of γ-glutamic acid using the DIC/HOBt method (with a 5 equivalent excess with respect to resin loading) in DMF. The mixture was rotated on a rotor at room temperature for 2 hours. Resin was filtered and washed with DMF/DCM/DMF (6×30 mL each). The Fmoc group on the γ-glutamic acid was de-protected by treating it twice with 20% (v/v) piperidine/DMF solution for 5 and 15 minutes (25 mL). The resin was washed with DMF/DCM/DMF (6:6:6 times each). A Kaiser tests performed on peptide resin aliquots upon completion of Fmoc-deprotection were positive.

Final cleavage of peptide from the resin:

The peptidyl resin synthesized by manual synthesis was washed with DCM (6×10 mL), MeOH (6×10 mL) and ether (6×10 mL) and dried in vacuum desiccators overnight. Cleavage of the peptide from the solid support was achieved by treating the peptide-resin with a reagent cocktail (80% TFA/5% thioanisole/5% phenol/2.5% EDT/2.5% DMS/5% DCM) at room temperature for 3 hours. Cleavage mixtures were collected by filtration and the resins were washed with TFA (2 mL) and DCM (2×5 mL). The excess TFA and DCM was concentrated to small volume under nitrogen and a small amount of DCM (5-10 mL) was added to the residue and evaporated under nitrogen. The process was repeated 3-4 times to remove most of the volatile impurities. The residue was cooled to 0° C. and anhydrous ether was added to precipitate the peptide. The precipitated peptide was centrifuged and the supernatant ether was removed and fresh ether was added to the peptide and re-centrifuged. The crude sample was preparative HPLC purified and lyophilized. The identity of peptides were confirmed by LCMS.

### Example 3: In Vitro Cellular Assay for Human FGF21 Receptor Efficacy in CHO Cells (in-Cell Western)

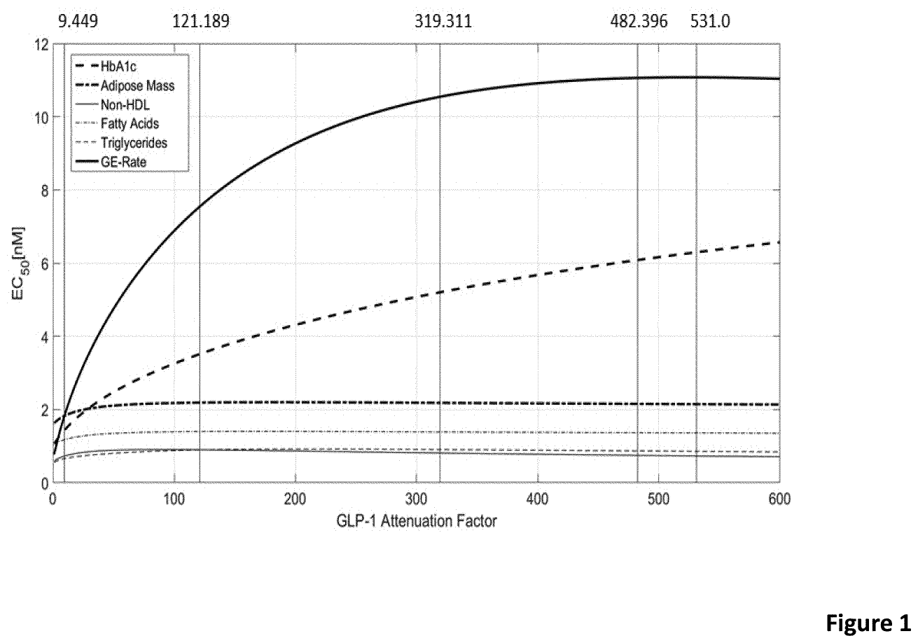

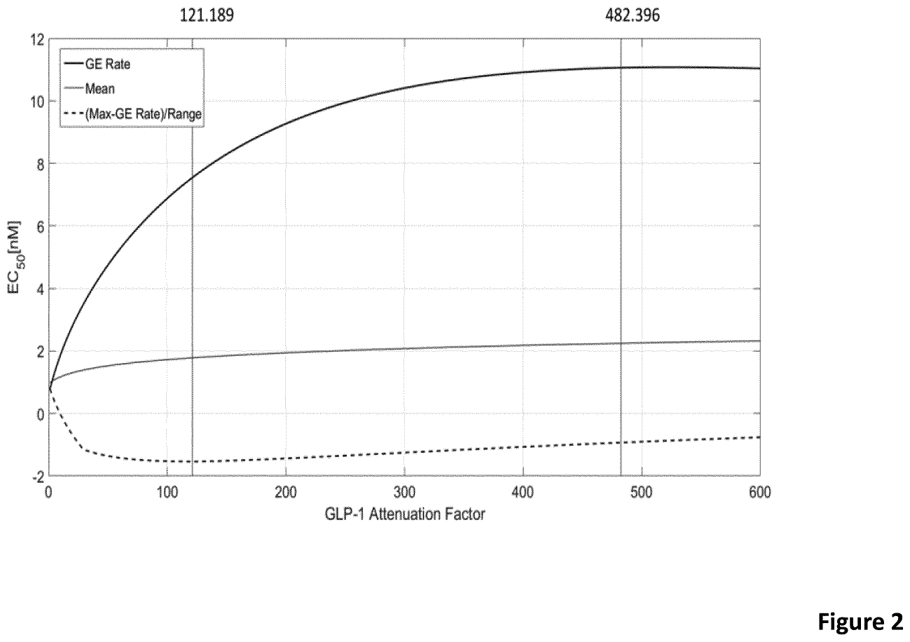

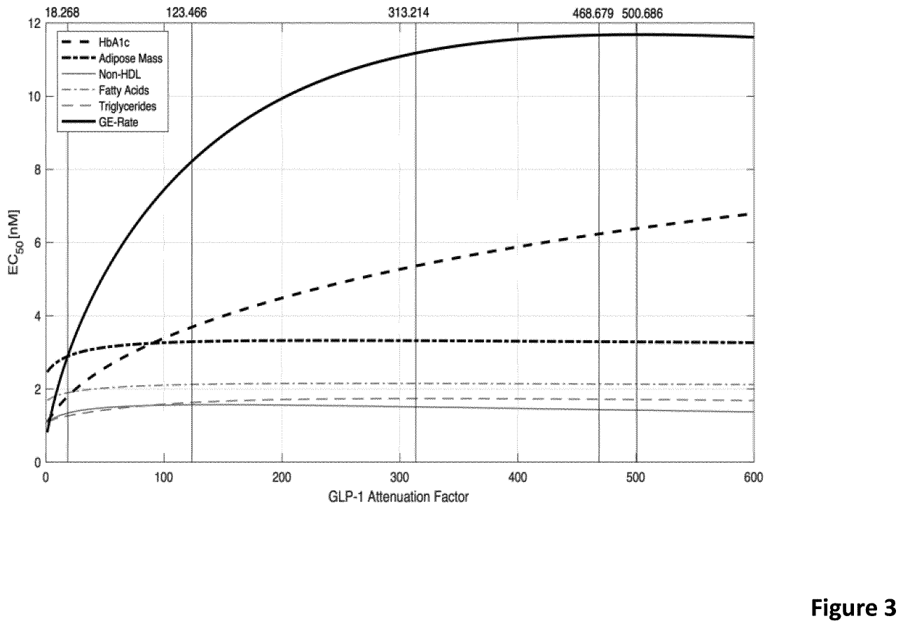

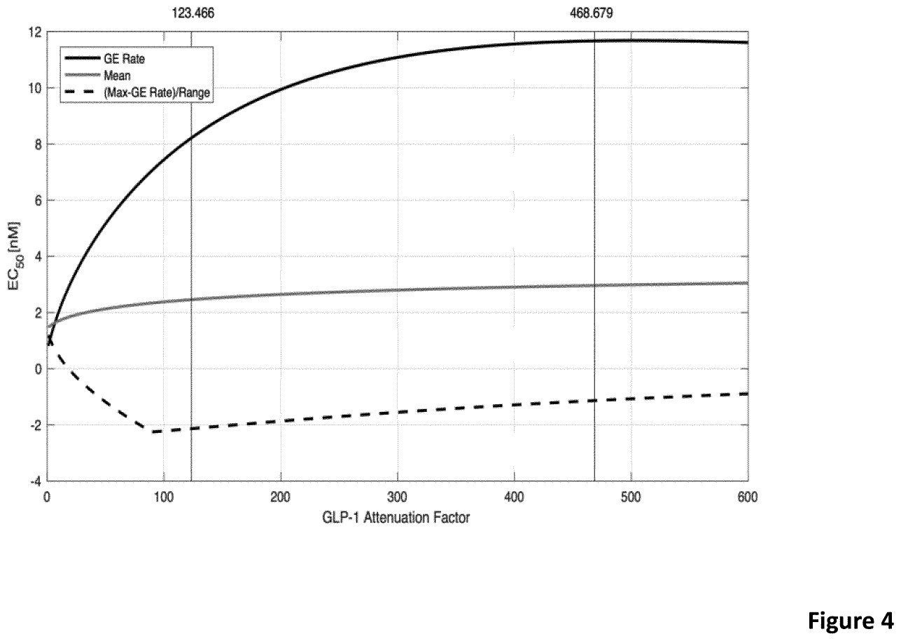

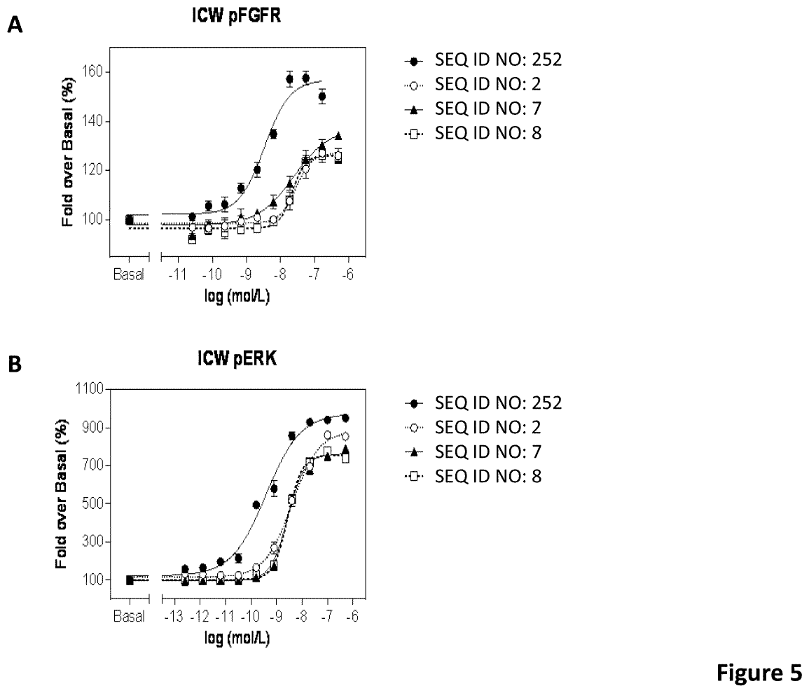

The cellular in vitro efficacy of G-FGF21 (SEQ ID NO: 252) and fusion proteins of the invention were measured using a specific and highly sensitive In-Cell Western (ICW) assay. The ICW assay is an immunocytochemical assay that is usually performed using a microplate format. CHO Flp-In cells (Invitrogen, Darmstadt, Germany) stably expressing the human FGFR1c together with human beta-Klotho (KLB) were used for an FGF21 receptor autophosphorylation ICW assay (Aguilar et al. (2010) PLoS ONE 5(4): e9965). In order to determine the receptor autophosphorylation level or downstream activation of the MAP kinase ERK1/2, 2×10⁴ cells/well were seeded into 96-well plates and grown for 48 h. Cells were serum starved with serum-free medium Ham's F-12 Nutrient Mix with GlutaMAX (Gibco, Darmstadt, Germany) for 3-4 hours. The cells were subsequently treated with increasing concentrations of either G-FGF21 (SEQ ID NO: 252) or the indicated fusion protein for 5 minutes at 37° C. After incubation, the medium was discarded, and the cells were fixed in 3.7% freshly prepared para-formaldehyde for 20 minutes. Cells were permeabilized with 0.1% Triton-X-100 in PBS for 20 minutes. Blocking was performed with Odyssey blocking buffer (LICOR, Bad Homburg, Germany) for 2 hours at room temperature. As primary antibody, anti-pFGFR Tyr653/654 (New England Biolabs, Frankfurt, Germany) or anti-pERK Phospho-p44/42 MAP Kinase Thr202/Tyr204 (Cell Signaling) was added and incubated overnight at 4° C. After incubation of the primary antibody, cells were washed with PBS plus 0.1% Tween20. The cells were then incubated with secondary anti-Mouse 800CW antibody (LICOR, Bad Homburg, Germany) for 1 hour at room temperature. Subsequently, the cells were washed again with PBS plus 0.1% Tween20. Infrared dye signals were quantified with an Odyssey imager (LICOR, Bad Homburg, Germany). Results were normalized by quantification of DNA with TO-PRO3 dye (Invitrogen, Karlsruhe, Germany). Data were obtained as arbitrary units (AU), and EC50 values were obtained from dose-response curves (summarized in Tables 2 and 3). FIG. 5 shows the results from an ICW with CHO cells overexpressing human FGFR1c plus KLB.

TABLE 2
EC50 values of G-FGF21 (SEQ ID NO: 252) and GLP-1RA/FGF21
Fc fusion proteins measured via ICW pFGFR in CHO cells.

| | pFGFR ICW |
|---|---|
| SEQ ID NO | EC50 (nmol/L) |
| 252 | 4.49 |
| 1 | 38.61 |
| 2 | 30.57 |
| 3 | 22.72 |
| 4 | 36.35 |
| 5 | 20.38 |
| 6 | 19.73 |
| 7 | 27.85 |
| 8 | 25.39 |
| 19 | 34.31 |
| 20 | 42.86 |
| 23 | 54.56 |
| 24 | 25.29 |
| 39 | 13.85 |
| 44 | 29.99 |
| 57 | 25.83 |
| 71 | 13.57 |
| 83 | 16.60 |
| 209 | 66.29 |

TABLE 3
EC50 values of G-FGF21 (SEQ ID NO: 252) and GLP-1RA/FGF21
Fc fusion proteins measured via ICW pERK in CHO cells.

| | pERK ICW |
|---|---|
| SEQ ID NO | EC50 (nmol/L) |
| 252 | 0.17 |
| 1 | 6.72 |
| 2 | 6.94 |
| 3 | 6.07 |
| 4 | 8.61 |
| 5 | 6.84 |
| 6 | 9.86 |
| 7 | 5.96 |
| 8 | 6.75 |
| 19 | 43.75 |
| 20 | 10.40 |
| 23 | 8.60 |
| 24 | 8.39 |
| 39 | 4.28 |
| 44 | 5.40 |
| 57 | 6.86 |
| 71 | 6.19 |
| 83 | 2.44 |
| 209 | 27.93 |

### Example 4: In Vitro Cellular Assay for Human Glucagon-Like-Peptide 1 (GLP-1) Receptor Efficacy

Agonism of compounds for human glucagon-like peptide-1 (GLP-1) receptor was determined by functional assays measuring the cAMP responses in a HEK-293 cell line stably expressing human GLP-1 receptor.

Recombinant HEK-293 cells were grown in T175 culture flasks placed at 37° C. to near confluence in medium (DMEM with 10% FBS) and collected in 2 mL vials in cell culture medium containing 10% DMSO in concentrations of 1-5×10⁷ cells/mL. Each vial contained 1.8 mL cell suspension. The vials were slowly frozen to −80° C. in an isopropanol chamber, and then transferred to liquid nitrogen for long term storage.

Prior to their use, frozen cells were thawed quickly at 37° C., washed with 20 mL cell buffer (1×HBSS; 20 mM HEPES, 0.1% BSA) and centrifuged for 5 minutes at 900 rpm. Cells were resuspended in assay buffer (cell buffer plus 2 mM IBMX) and adjusted to a cell density of 1×10⁶ cells/mL. For measurement, 5 μL cell suspension (final 5×10³ cells/well) and 5 μL of test compound were added to a well of a 384-well plate, followed by incubation for 30 minutes at room temperature. Human GLP-1(7-36) amide from Bachem (Bubendorf, Switzerland, H-6795) (SEQ ID NO: 260) was taken as a control. The cAMP content of cells was determined using a kit from Cisbio Corp. (cat. no. 62AM4PEC) based on Homogenous Time Resolved Fluorescence (HTRF). After addition of HTRF reagents diluted in lysis buffer (kit components), the plates were incubated for 1 h, followed by measurement of the fluorescence ratio at 665/620 nm. The li vitro potencies of agonists were quantified by determining the concentrations that caused 50% activation of maximal response (EC50).

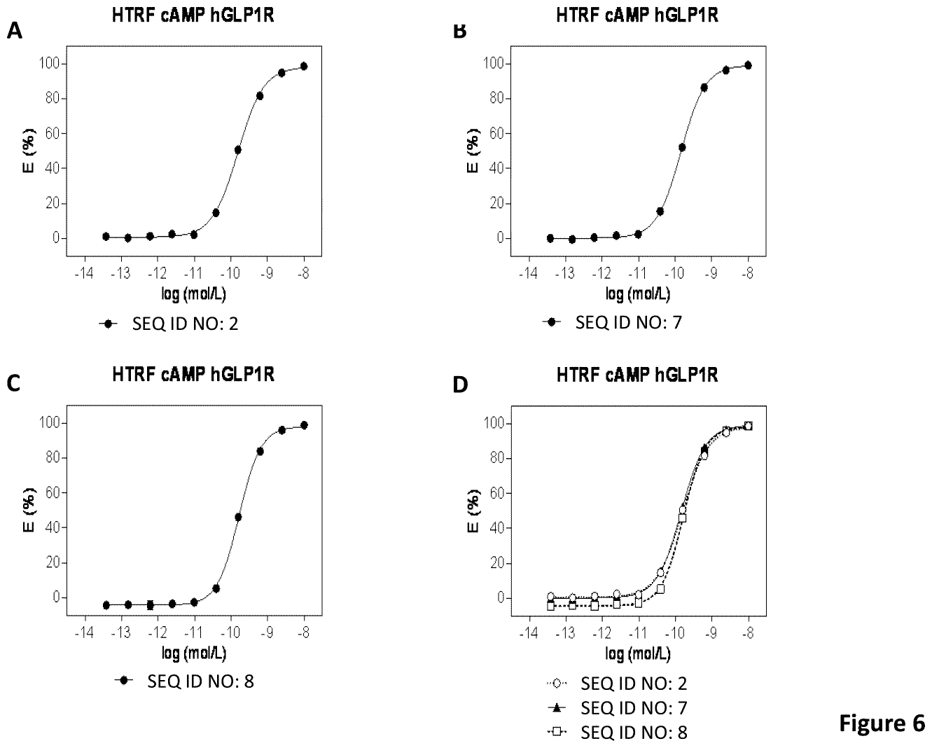

The results are summarized in Table 4, and dose-response curves are shown in FIG. 6.

TABLE 4
EC50 values of human GLP-1(7-36) (SEQ ID NO: 260), GLP-1RA/
FGF21 Fc fusion proteins and several single GLP-1R agonistic
peptides measured via HTRF cAMP assay in HEK-293 cells.

| | HTRF cAMP EC50 (pmol/L) | | |
|---|---|---|---|
| | Human | Mouse | Monkey |
| SEQ ID NO | GLP-1R | GLP-1R | GLP-1R |
| 260 | 0.77 | 0.62 | 1.12 |
| 1 | 218.76 | 2.80 | 161.07 |
| 2 | 193.72 | 2.93 | 164.36 |
| 3 | 211.94 | 97.21 | 278.82 |
| 4 | 313.33 | 5.29 | 261.98 |
| 5 | 125.01 | 96.46 | 195.69 |
| 6 | 383.35 | 6.45 | 347.21 |
| 7 | 130.91 | 68.11 | 190.20 |
| 8 | 152.24 | 77.14 | 217.09 |
| 18 | 9.90 | 2.09 | 10.18 |
| 19 | 11.20 | 2.16 | 10.95 |
| 20 | 9.49 | 1.97 | 9.86 |
| 21 | 93.80 | 4.19 | 78.68 |
| 22 | 85.58 | 3.88 | 73.93 |
| 23 | 78.72 | 3.32 | 70.72 |
| 24 | 48.83 | 2.66 | 43.54 |
| 25 | 45.94 | 101.49 | 81.30 |
| 26 | 40.08 | 95.33 | 73.30 |
| 27 | 37.53 | 90.96 | 75.10 |
| 28 | 32.85 | 106.99 | 68.70 |
| 29 | 37.67 | 113.45 | 64.00 |
| 30 | 41.96 | 125.49 | 73.30 |
| 31 | 32.31 | 113.47 | 62.80 |
| 39 | 8.50 | 1.16 | 12.30 |
| 40 | 46.06 | 39.02 | 50.40 |
| 42 | 18.07 | 43.21 | 22.20 |
| 43 | 13.69 | 31.48 | 17.40 |
| 44 | 125.83 | 325.07 | 174.00 |
| 45 | 48.20 | 492.12 | 60.42 |
| 46 | 8.47 | 17.85 | 11.40 |
| 47 | 16.99 | 6.64 | 35.60 |
| 48 | 35.70 | 10.07 | 60.60 |
| 49 | 23.37 | 6.65 | 42.20 |
| 50 | 20.51 | 7.87 | 38.20 |
| 51 | 26.84 | 7.58 | 51.10 |
| 52 | 25.60 | 7.04 | 46.00 |
| 53 | 28.37 | 128.18 | 25.03 |
| 54 | 107.04 | 118.79 | 97.40 |
| 55 | 261.38 | 595.82 | 156.56 |
| 56 | 15.02 | 66.73 | 11.48 |
| 57 | 21.12 | 19.29 | 15.70 |
| 58 | 9.38 | 7.19 | 5.76 |
| 59 | 13.64 | 13.50 | 10.31 |
| 60 | 22.52 | 11.72 | 13.02 |
| 61 | 68.26 | 42.99 | 40.20 |
| 62 | 20.32 | 13.38 | 12.93 |
| 63 | 95.38 | 54.34 | 52.60 |
| 64 | 9.09 | 6.08 | 7.47 |
| 65 | 16.49 | 6.63 | 10.17 |
| 66 | 33.77 | 11.02 | 31.42 |
| 67 | 36.52 | 10.23 | 21.65 |
| 68 | 16.66 | 9.42 | 10.80 |
| 69 | 15.30 | 17.28 | 14.69 |
| 70 | 41.39 | 83.11 | 38.80 |
| 71 | 27.79 | 53.55 | 26.61 |
| 72 | 20.37 | 77.53 | 25.04 |
| 74 | 13.47 | 18.34 | 13.70 |
| 76 | 39.21 | 189.87 | 40.32 |
| 78 | 50.98 | 323.16 | 52.52 |
| 79 | 243.25 | 2512.19 | 295.89 |
| 80 | 48.63 | 64.39 | 42.55 |
| 81 | 49.87 | 67.40 | 49.36 |
| 82 | 90.13 | 72.47 | 109.18 |
| 83 | 29.30 | 55.99 | 28.11 |
| 84 | 38.20 | n.d. | n.d. |
| 88 | 33.03 | n.d. | n.d. |
| 89 | 45.29 | n.d. | n.d. |
| 90 | 336.00 | n.d. | n.d. |
| 92 | 58.86 | n.d. | n.d. |
| 93 | 48.35 | n.d. | n.d. |
| 94 | 66.49 | n.d. | n.d. |
| 95 | 18.49 | n.d. | n.d. |
| 96 | 43.99 | n.d. | n.d. |
| 97 | 40.39 | n.d. | n.d. |
| 100 | 15.01 | n.d. | n.d. |
| 101 | 83.50 | n.d. | n.d. |
| 102 | 169.50 | n.d. | n.d. |
| 105 | 8.12 | n.d. | n.d. |
| 106 | 9.66 | n.d. | n.d. |
| 107 | 186.50 | n.d. | n.d. |
| 108 | 326.00 | n.d. | n.d. |
| 109 | 278.00 | n.d. | n.d. |
| 112 | 7.23 | n.d. | n.d. |
| 113 | 158.50 | n.d. | n.d. |
| 115 | 92.20 | n.d. | n.d. |
| 116 | 10.61 | n.d. | n.d. |
| 118 | 18.27 | n.d. | n.d. |
| 120 | 10.17 | n.d. | n.d. |
| 121 | 11.87 | n.d. | n.d. |
| 122 | 14.53 | n.d. | n.d. |
| 123 | 12.74 | n.d. | n.d. |
| 124 | 10.58 | n.d. | n.d. |
| 126 | 8.51 | n.d. | n.d. |
| 127 | 10.27 | n.d. | n.d. |
| 128 | 9.83 | n.d. | n.d. |
| 129 | 12.30 | n.d. | n.d. |
| 130 | 7.38 | n.d. | n.d. |
| 132 | 8.48 | n.d. | n.d. |
| 133 | 13.44 | n.d. | n.d. |
| 134 | 17.34 | n.d. | n.d. |
| 135 | 11.49 | n.d. | n.d. |
| 136 | 11.84 | n.d. | n.d. |
| 139 | 7.35 | n.d. | n.d. |
| 142 | 12.89 | n.d. | n.d. |
| 143 | 14.20 | n.d. | n.d. |
| 144 | 16.22 | n.d. | n.d. |
| 145 | 7.63 | n.d. | n.d. |
| 146 | 12.25 | n.d. | n.d. |
| 147 | 10.11 | n.d. | n.d. |
| 148 | 8.48 | n.d. | n.d. |
| 150 | 11.53 | n.d. | n.d |
| 151 | 8.66 | n.d. | n.d. |
| 152 | 9.43 | n.d. | n.d. |
| 153 | 9.98 | n.d. | n.d. |
| 155 | 11.02 | n.d. | n.d. |
| 156 | 9.22 | n.d. | n.d. |
| 157 | 12.08 | n.d. | n.d. |
| 158 | 8.70 | n.d. | n.d. |
| 161 | 252.21 | n.d. | n.d. |
| 162 | 270.04 | n.d. | n.d. |
| 163 | 374.50 | n.d. | n.d. |
| 164 | 102.76 | 87.97 | 141.14 |
| 165 | 173.00 | n.d. | n.d. |
| 166 | 159.50 | n.d. | n.d. |
| 167 | 309.50 | n.d. | n.d. |
| 168 | 46.10 | n.d. | n.d. |
| 169 | 59.60 | n.d. | n.d. |
| 170 | 233.50 | n.d. | n.d. |
| 171 | 266.50 | n.d. | n.d. |
| 172 | 253.50 | n.d. | n.d. |
| 174 | 76.95 | n.d. | n.d. |
| 175 | 90.85 | n.d. | n.d. |
| 176 | 71.75 | n.d. | n.d. |
| 177 | 48.65 | n.d. | n.d. |
| 180 | 405.50 | n.d. | n.d. |
| 181 | 137.00 | n.d. | n.d. |
| 182 | 123.00 | n.d. | n.d. |
| 183 | 73.80 | n.d. | n.d. |
| 184 | 216.00 | n.d | n.d. |
| 185 | 13.85 | n.d. | n.d. |
| 186 | 123.85 | n.d. | n.d. |
| 187 | 67.44 | 67.47 | 63.20 |
| 188 | 59.65 | 70.96 | 63.70 |
| 190 | 152.41 | 479.17 | 238.78 |
| 192 | 102.27 | 98.00 | 148.81 |
| 193 | 108.18 | 71.30 | 151.49 |
| 194 | 127.68 | 129.00 | 139.93 |
| 195 | 74.51 | 56.60 | 71.84 |
| 196 | 75.28 | 31.70 | 126.80 |
| 197 | 68.32 | 48.00 | 101.65 |
| 198 | 86.54 | 65.10 | 86.91 |
| 199 | 163.23 | 153.00 | 231.19 |
| 200 | 73.11 | 46.20 | 110.64 |
| 201 | 100.55 | 69.80 | 98.56 |
| 202 | 100.50 | 71.70 | 127.68 |
| 203 | 96.88 | 89.20 | 101.69 |
| 204 | 102.74 | 65.70 | 93.49 |
| 205 | 130.74 | 127.00 | 113.32 |
| 206 | 107.18 | 93.70 | 150.82 |
| 207 | 146.88 | 128.00 | 188.80 |
| 208 | 169.64 | 127.95 | 254.61 |
| 209 | 164.10 | 118.21 | 231.66 |
| 211 | 58.80 | 151.66 | 68.55 |
| 212 | 113.42 | 286.97 | 153.33 |
| 216 | 151.60 | 346.64 | 262.81 |
| 217 | 7.62 | 5.39 | n.d. |
| 219 | 221.50 | 422.46 | n.d. |
| 220 | 95.55 | 110.49 | n.d. |
| 221 | 202.49 | 162.48 | n.d. |
| 222 | 327.48 | 200.50 | n.d. |
| 223 | 80.85 | 79.52 | n.d. |
| 224 | 226.96 | 250.98 | n.d. |
| 225 | 127.98 | 61.60 | n.d. |
| 226 | 64.09 | 43.53 | n.d. |
| 227 | 61.35 | 40.75 | n.d. |
| 228 | 49.19 | 30.10 | n.d. |
| 229 | 62.87 | 469.79 | 82.08 |
| 261 | 56.32 | 1.12 | 49.40 |
| 262 | 10.48 | 7.65 | 5.74 |
| 271 | 6.94 | 0.84 | 9.51 |
| 272 | 20.80 | 1.31 | 21.74 |
| 274 | 19.47 | 71.25 | 19.95 |
| 275 | 31.15 | 101.21 | 20.00 |
| 278 | 10.48 | 7.65 | 5.74 |
| 279 | 210.23 | 380.23 | 250.95 |
| 281 | 9.87 | 20.04 | 7.11 |
| 282 | 34.04 | 40.15 | 26.60 |
| 284 | 51.40 | 69.52 | 39.71 |
| 286 | 15.57 | 29.08 | 12.99 |
| 291 | 28.89 | 11.09 | 28.36 |
| 292 | 8.48 | 8.56 | 8.77 |
| 293 | 29.23 | 5.06 | 27.60 |
| 294 | 208.98 | 42.34 | 182.99 |
| 295 | 9.23 | 49.40 | 9.87 |
| 297 | 21.30 | 33.10 | 21.54 |
| 298 | 93.60 | 65.80 | 56.70 |
| 301 | 23.68 | 23.70 | 16.31 |

n.d.: not determined

### Example 5: Analyzing Conformational and Thermal Stability of GLP-1RA/FGF21 Fc Fusion Proteins

Conformational stability and propensity to aggregate was determined simultaneously for the GLP-1RA/FGF21 Fc fusion proteins using a UNit (Unchained Labs, Calif., USA). The UNit combines the analysis of intrinsic fluorescence of a protein or polypeptide to detect unfolding of the protein or polypeptide and a Static Light Scattering (SLS) measurement in order to investigate aggregation behavior.

Data were acquired for the fusion proteins at a concentration of 5 mg/mL formulated in phosphate buffer at pH 7.4. A volume of 9 μL of each sample were loaded into a UNi capillary holder and analyzed in triplicate on the UNit. The temperature was increased from 20 to 95° C. at a constant linear rate of 0.3° C./minutes. The BaryCentric Mean (BCM) indicating the intrinsic fluorescence and SLS signals detected with a 266 nm laser were plotted against the applied temperature in order to obtain melting temperatures (Tm) and aggregation onset temperatures (Tagg). Data were analyzed using the UNit Analysis software v. 2.1 and are summarized in Table 5.

In addition, for some proteins a thermal shift assay was applied to analyze thermostability in imitation of differential scanning fluorimetry (DSF or ThermoFluor™) assays (Ahmad S. et al. (2012) Protein Science 21: 433-446; Pantoliano et al. (2001) J. Biomol. Screen 6: 429-440; Niesen et al. (2007) Nat. Protoc. 2: 2212-21). This assay is based on the observation that hydrophobic fluorescent dyes, such as Sypro™ Orange (Life Technologies, cat. No. S6651), increase their fluorescence when they bind to hydrophobic patches on a protein. Such hydrophobic patches are exposed in proteins when they unfold upon heating, so that the increase in fluorescence can be used as a measure for the degree of unfolding and, hence, for the thermostability of the proteins.

Proteins were tested by mixing a solution of each protein in PBS (Gibco) with a 160× solution of Sypro™Orange (diluted in water from a 5000×DMSO stock as provided by the supplier). The sample volume was adjusted to 20 μL with PBS. Typical conditions contained 0.8 mg/mL protein and 8× Sypro™Orange in the final mixture, but protein concentrations could be varied between 0.4 mg/mL and 1.2 mg/mL. Samples were dispensed in 96-well PCR plates (BioRad Semi-Skirt 96 white) and shortly centrifuged to remove air bubbles. Plates were inserted in a BioRad iQ5 real-time PCR instrument and subjected to a thermal gradient from 10 to 90° C. at a ramp speed of 1° C./minute. For excitation and quantification of fluorescence, filters for wavelengths of 485 nm and 575 nm were chosen. BioRad iQ5 Standard Edition software (v. 2.0.148.60623) was used for data processing. In curves of fluorescence intensity against temperature, the inflection point was chosen as the measure for the melting temperature (Tm).

TABLE 5
Melting and aggregation temperatures of G-FGF21 (SEQ ID NO: 252)
and selected GLP-1RA/FGF21 Fc fusion proteins.

| | Tm | Tagg |
|---|---|---|
| SEQ ID NO | (° C.) | (° C.) |
| 252 | 43.1* | n.d. |
| 2 | 69.6 | 71.5 |
| 7 | 68.7 | 66.1 |
| 8 | 68.6 | 65.3 |
| 4 | 64.9 | 64.0 |
| 6 | 64.1 | 63.4 |
| 3 | 63.9 | 65.3 |
| 5 | 62.6 | 63.2 |
| 1 | 70.5 | 75.5 |

*Data generated via DSF; n.d .: not determined

### Example 6: Pharmacokinetics in Mouse and Non-Human Primate

Plasma concentrations and pharmacokinetic parameters of GLP-1 RA/FGF21 Fc fusion proteins were determined after single subcutaneous administration of 0.3 mg/kg in solution to female C57BI/6 mice or male Cynomolgus monkeys using three different methods. Blood samples were obtained at time points from 30 minutes to 168 hours after dosing.

a.) Bioanalytical Screening Method for Quantification of the Intact FGF21 Part of the GLP-1 RA/FGF21 Fc Fusion Proteins

Plasma samples were analyzed for the intact FGF21 part of the fusion proteins with an ELISA kit (F1231-K01, Eagle Biosciences, USA). The assay utilized the two-site sandwich technique with two selected antibodies that bound to different epitopes of human intact FGF21. One of the antibodies specifically bounds to the N-terminal amino acids (aa) 29-35 of human FGF21 and the other antibody specifically bound to the C-terminus (aa 203-209) of human FGF21. Assay standards, controls and unknown samples were added directly to wells of microplate that is coated with an anti-human FGF21 (aa 29-35)-specific antibody. Simultaneously, a horseradish peroxidase conjugated anti-human FGF21 (aa 203-209)-specific antibody was added to each well. After the first incubation period, the antibody on the walls of microtiter wells captured human FGF21 in the sample and unbound protein in each microtiter well is washed away. A “sandwich” of “anti-FGF21 antibody-human intact FGF21-HRP conjugated tracer antibody” was formed. The unbound tracer antibody was removed in the subsequent washing step. For the detection of this immunocomplex, the well was then incubated with a substrate solution in a timed reaction and then measured in a spectrophotometric microplate reader. The enzymatic activity of the immunocomplex bound to human intact FGF21 on the walls of the microtiter wells was directly proportional to the amount of intact FGF21 in the sample.

b.) Bioanalytical Screening Method for Quantification of the Intact Full-Length Fusions Proteins

The concentration of the full-length GLP-1RA/FGF21 Fc fusion proteins in plasma were determined utilizing an ELISA method. The N-terminus of the fusion proteins were captured by a mouse monoclonal anti-GLP1 antibody (Mesoscale Discovery, MSD). Following blocking the plates with 150 μL of Blocker A (MSD) for 1 hour at room temperature (RT) with gentle shaking and washing 3 times with 300 μL wash buffer 50 μL of diluted plasma samples (standards and PK study samples) were added to each well and the plates were incubated for 1 hour at RT with gentle shaking. Following washing 3 times with 300 μL wash buffer 50 μL of the primer detection antibody (C-terminus rabbit anti-FGF21 antibody, Pineda Antikörper-Service, Berlin, Germany) was added to each well and the plates were incubated for 1 hour at RT. Following washing 3 times with 300 μL wash buffer 25 μL goat-anti-rabbit antibody (Sulfo-Tag labelled, MSD) diluted in PBS-Tween 0.05% (PBS-T) was added to each well and the plates were incubated for 1 hour at RT. Following washing 3 times with 300 μL PBS-T 150 μL read buffer was added to the wells.

c.) Bioanalytical Screening Method for Quantification of the Intact GLP-1 Part of the GLP-1 FGF21 Fc Fusion Proteins

Plasma samples were analyzed for the intact GLP-1 part of the fusion proteins with a GLP-1 ELISA method. The ELISA plates were coated with mouse monoclonal anti-GLP-1 antibody (Mesoscale Discovery, MSD). Following blocking with 150 μL of Blocker A (MSD) for 1 hour at room temperature (RT) with gentle shaking and washing 3 times with 300 μL PBS-T, 50 μL of diluted plasma samples (standards and PK study samples) were added to each well and the plates were incubated for 1 hour at RT with gentle shaking. Following washing 3 times with 300 μL PBS-T, 25 μL of goat anti-human IgG diluted ( 1/3,333) in PBS-T (Sulfo-Tag labelled, MSD) was added to each well and the plates were incubated for 1 hour at RT. Following washing 3 times with 300 μL PBS-T, 150 μL read buffer was added to the wells.

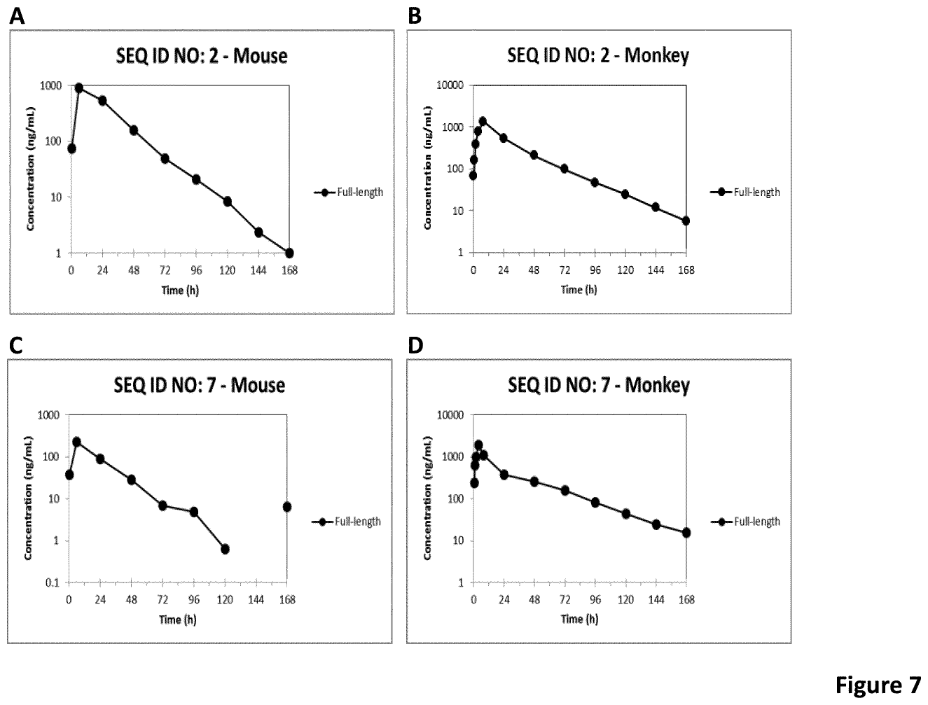

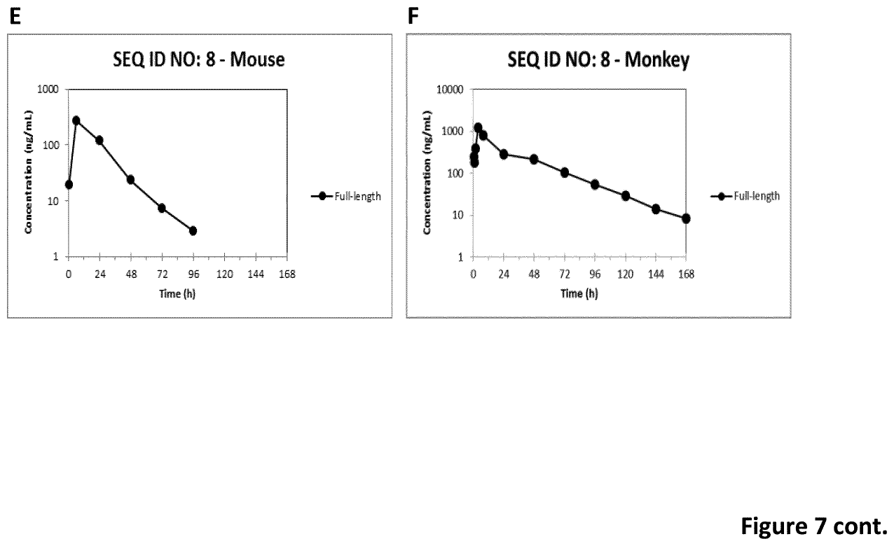

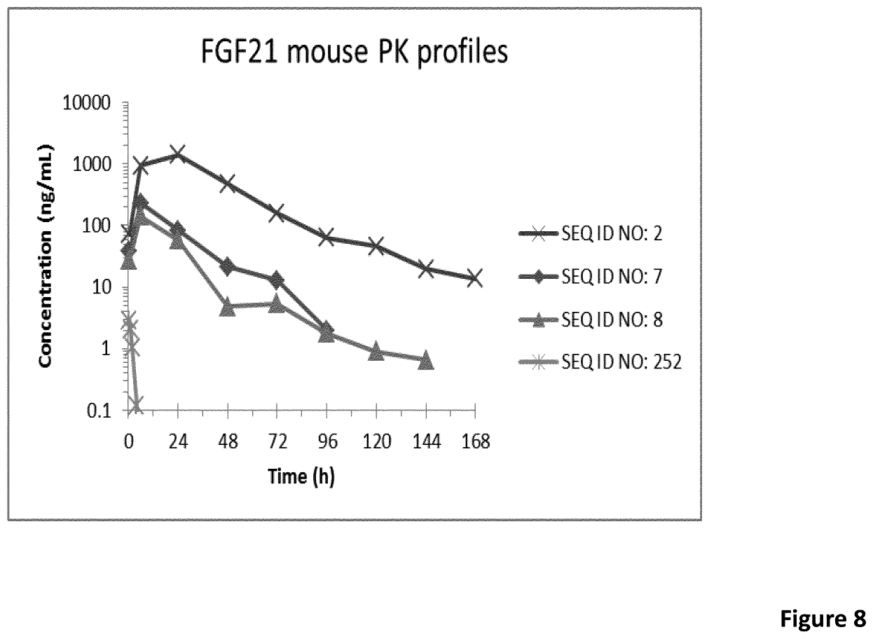

The pharmacokinetic parameters were calculated by the program WinNonlin 6.4 using a non-compartmental model and linear trapezoidal interpolation calculation. The results are presented in FIGS. 7 and 8 as well as in Table 6. The results show that the novel GLP-1 RA/FGF21 Fc fusion proteins maintained their plasma levels in the ng/mL range with half-lives up to 20-40 hours.

TABLE 6
Terminal half-lives of selected GLP-1RA/FGF21 Fc fusion proteins
and G-FGF21 (SEQ ID NO: 252) after subcutaneous injection of
0.3 mg/kg in mouse and non-human primate.

| | | t_{1/2} mouse | t_{1/2} monkey |
|---|---|---|---|
| SEQ ID NO | Assay | (h) | (h) |
| 2 | FGF21 intact | 27.6 | 42.5 |
| | GLP-1 intact | 27.8 | 33.4 |
| | full-length | 16.1 | 23.5 |
| 7 | FGF21 intact | 14.2 | 31.7 |
| | GLP-1 intact | n.d. | 21.3 |
| | full-length | 13.9 | 28.7 |
| 8 | FGF21 intact | 17.9 | 33.6 |
| | GLP-1 intact | n.d. | 23.7 |
| | full-length | 13.3 | 27.6 |
| 252 | FGF21 intact | 0.7 | n.d. |
| | GLP-1 intact | n.d. | n.d. |
| | full-length | n.d. | n.d. |

n.d.: not determined

### Example 7: In Vivo Efficacy in Murine Models

a.) Multiple Dose Diet-Induced Obese (DIO) Mice

Female C57BL/6N Charles River mice were housed in groups in a specific pathogen-free barrier facility on a 12 hour light/12 hour dark cycle with free access to water and standard or high-fat diet (ssniff adjusted Fat Diet E15797). After 20 weeks of pre-feeding on high-fat diet, mice were stratified for body weight to treatment groups (n=8), so that each group had similar mean body weight. An age-matched group with ad-libitum access to standard chow (ssniff R/M-H, V1534-0) was included as standard control group. A dulaglutide treated group was also included as comparator. Prior to the start of treatment, mice were subcutaneously (s.c.) injected with vehicle solution and weighed for 3 days to acclimate them to the procedures.

1) Acute effect on blood glucose in fed female DIO mice: initial blood samples were taken just before first administration (s.c.) of vehicle (phosphate buffer solution) or first administration of GLP-1 RA/FGF21 Fc fusion proteins (dissolved in phosphate buffer), respectively. The volume of administration was 5 or 10 mL/kg, depending on concentration of stock solution. The animals had access to water and their corresponding diet during the experiment. Blood glucose levels were measured at t=0 hours, t=1 hour, t=2 hours, t=3 hours, t=4 hours, t=6 hours and t=24 hours (method: Accu-Check glucometer). Blood sampling was performed by tail incision without anaesthesia.

2) Chronic effect on body weight in female DIO mice: mice were treated once weekly, every 8^{th} day in the morning at the beginning of the light phase with either vehicle or test compounds for 4 weeks. Body weight and food consumption were recorded daily. Two days before start of treatment and on day 26, total fat mass was measured by nuclear magnetic resonance (NMR).

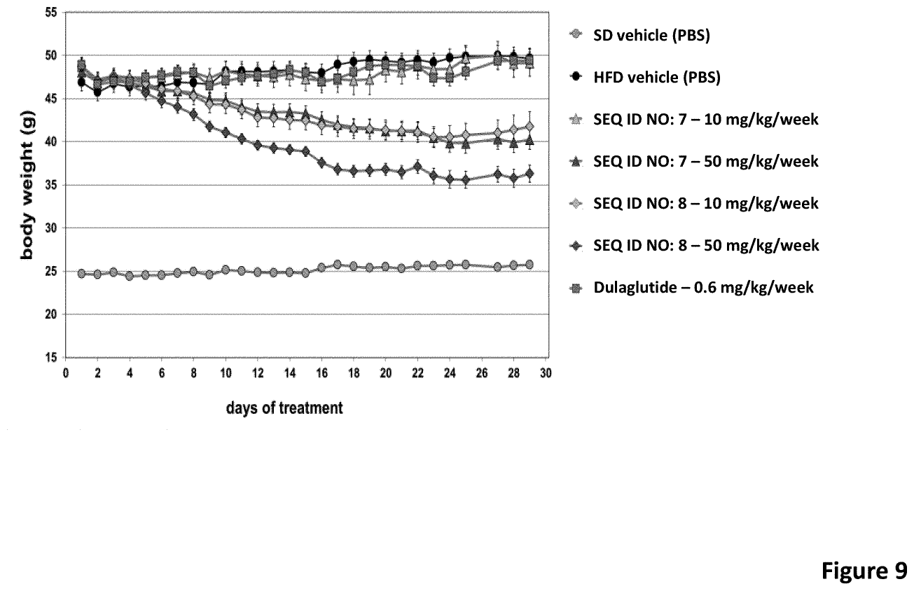

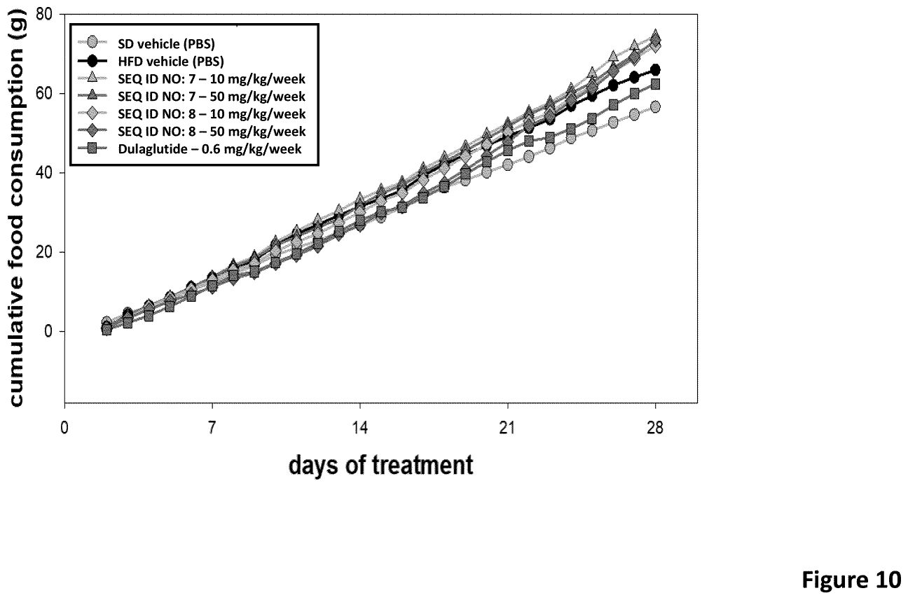

The effect of the fusion proteins on body weight and food consumption is shown in FIG. 9 and FIG. 10, respectively. Although animals treated with the fusion protein of SEQ ID NO: 8 or SEQ ID NO: 7 cumulatively consumed more food than vehicle or dulaglutide treated animals by the end of the study, they significantly lost more body weight than the vehicle or dulaglutide treated animals. This clearly demonstrates the balancing of the GLP-1 receptor activity versus the FGF21 mimetic activity of both molecules of SEQ ID NO: 7 and SEQ ID NO: 8 because their effect on body weight reduction did not need suppression of food consumption in order to materialize.

b.) Blood Glucose-Lowering Effects of Multiple, Subcutaneous Doses in Fed, Female Diabetic db/db Mice

Animals, Study Design (Pre-Dosing Phase, Dosing Phase), Pharmacological Intervention

Female, healthy, lean (BKS.Cg-(lean)/OlaHsd or BKS.Cg-Dock7(m)+/+Lepr(db)J) and diabetes-prone, obese db/db (BKS.Cg-+Leprdb/+Leprdb/OlaHsd or BKS.CG-m+/+Lepr(db)/J) mice were ordered from Envigo RMS Inc. or Charles River Laboratories. All animals were group housed in shoebox cages with wood chip bedding and were acclimated for approximately 2 to 3 weeks prior to dosing phase.

Mice were housed under vivarium conditions including a 12 hour light/12 hour dark cycle (light phase 04:00 AM-4:00 PM), a room temperature between 20-26° C. and a relative humidity between 30-70%. All animals had free access to Greenfield city water and a Purina Fomulab Diet 5008. At the study start mice were approximately 10-12 weeks old.

Pre-Dose Phase (15 Days)

Blood was collected for a HbA1c and blood glucose measurement on day 9 via tail clips. Blood glucose concentrations were measured using AlphaTRAK glucometers extended range (code 29 strips). Glucometer measurements were taken prior to any other in life activities and were performed in duplicate. If the values differed by more than 20 mg/dL (calculated glucometer value) a third value was recorded. Body mass measurements were collected on days 9 and 15. HbA1c and body mass values were used for block randomization. Animals were assigned to treatment groups (n=8/group) and to new cages and cage mates (n=4 animals/cage) on day 15 according to block randomization results. The lean group was included in the study as an age-matched, healthy reference group.

Dose Formulation and Dosing

Animals were either treated with a subcutaneous injection volume of 5 ml/kg of vehicle (sterile PBS), dulaglutide, SEQ ID NO: 8 or SEQ ID NO: 7 once on days 1, 8, 15, 22, and 27 of the dosing phase. Dosing was completed between 10:00 and 12:00 AM and was adjusted to the most recent body mass recording of each individual. Injection solutions including Trulicity (Dulaglutide Pen) were prepared by addition of sterile PBS to the stock solutions or Pen formulation in order to achieve the appropriate concentrations.

Dosinq Phase (36 Days)

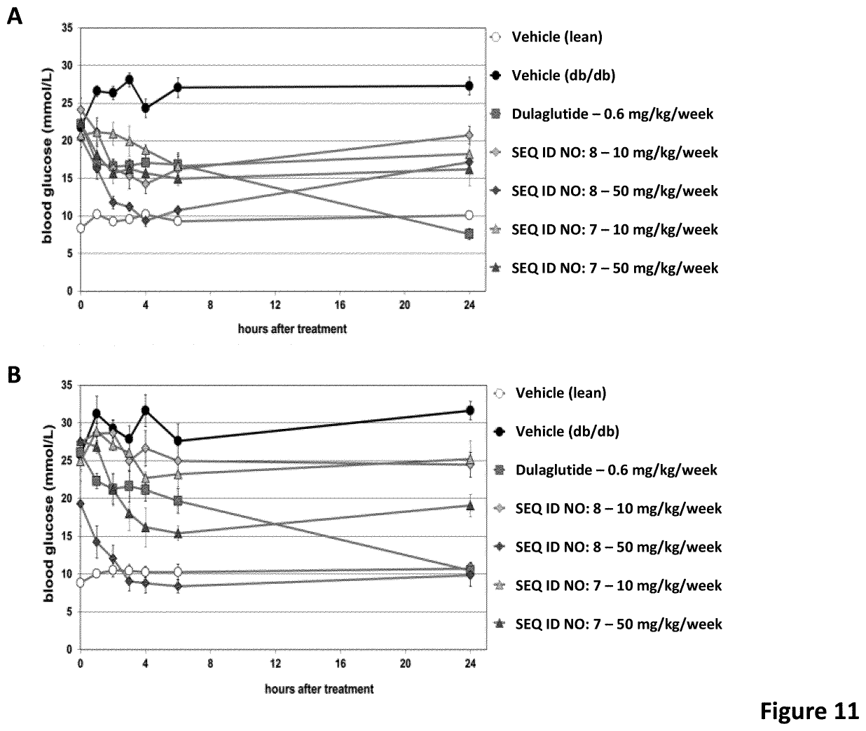

1) Blood glucose concentrations in morning-fed animals: Animals had unlimited access to water and feed during the experiment. Blood glucose was measured prior to any other in life activities between 10:00 to 12:00 AM on days 1, 2, 8, 9, 15, 16, 22, 23, 27, and 28 as well as 24 hours post-dose on days 2, 9, 16, 23, and 28. In addition, on days 1 and 22, blood was collected 1, 2, 3, 4, 6 and 24 hours post-dose (FIG. 11). Approximately 5 μL of blood were collected via tail clip and blood glucose measurements were performed in duplicate using AlphaTRAK glucometers with the extended range (code 29 strips). If the values differed by more than 20 mg/dL (calculated glucose value) then a third value was recorded. The Area Under the Curve (AUC) was calculated by the trapezoid method for each individual and time period as indicated.

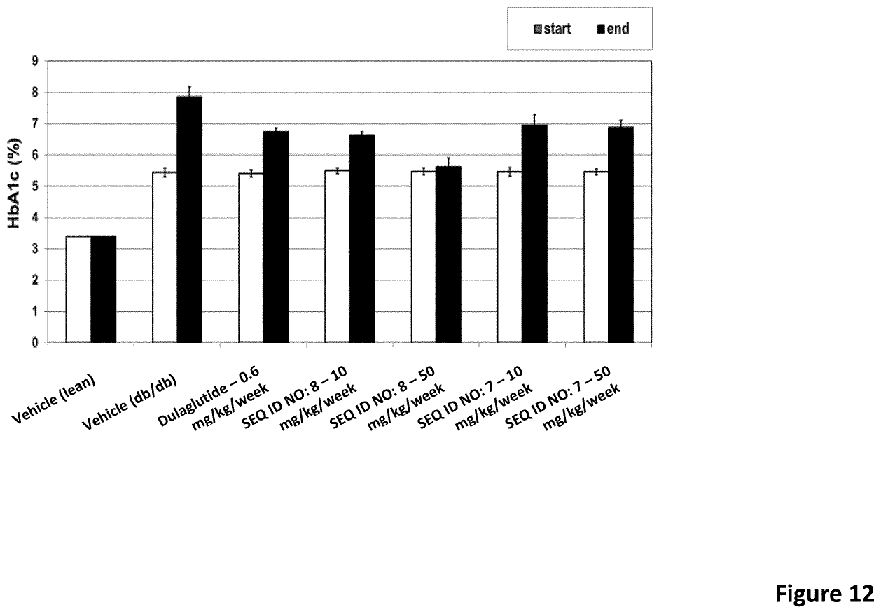

2) HbA1c analysis: Blood was collected via a tail clip on day 9 of the pre-dose phase and day 36 of the dosing phase. Blood was collected into a 5 μL non-additive micro capillary tube and immediately placed into a centrifuge tube containing hemolysate. The tube was shaken vigorously to mix the hemolysate with the blood and placed on a rocker to ensure the blood and reagent were completely mixed. Plasma HbA1c levels at study start and after study termination are shown in FIG. 12.

Statistical analyses: Data are depicted as means±SEM. For statistical analyses a One Way Analysis of Variance (ANOVA) and multiple comparisons (Dunnett's Method) were performed comparing the group of diabetic, obese db/db vehicle mice (n=8) with the group of diabetic, obese db/db test article treated mice (n=8). When the difference in the mean values of the two groups was greater than 0.05 they were considered statistically significantly different. Non-diabetic, lean-vehicle group data are depicted in FIGS. 11 and 12 and serve as a reference dataset for the non-obese, non-diabetic state.

In the animals treated with the fusion protein of SEQ ID NO: 8 or SEQ ID NO: 7, the lowering effect on blood glucose levels was significantly greater than in vehicle or dulaglutide treated animals (FIG. 11). The highest dose of the fusion protein of SEQ ID NO: 8 even lead to a reduction of blood glucose levels to normal non-diabetic animal levels over almost the whole 24 hour blood glucose profile measured on day 22 of treatment. In addition, animals treated with the fusion protein of SEQ ID NO: 8 or SEQ ID NO: 7 showed more pronounced suppression of HbA1c increase by the end of the study than vehicle or dulaglutide treated animals, as shown in FIG. 12.

c.) DIO-NASH Mouse Model

Animals and Experimental Set-Up

All animal experiments were conformed to international accepted principles for the care and use of laboratory animals.

Male C57BI/6J mice at 5 weeks of age were obtained from JanVier (JanVier labs, France), and each group housed 5 animals per cage under a 12 hour dark/12 hour light cycle. Room temperature was controlled to 22° C. 1° C., with 50%±10% humidity. Animals had ad libitum access to diet high in fat (40%, of these 18% trans-fat), 40% carbohydrates (20% fructose) and 2% cholesterol (D09100301, Research Diet, United States) previously described as the AMLN diet (Clapper et al. (2013) Am J Physiol Gastrointest Liver Physiol 305: G483-G495), or regular rodent chow (Altromin 1324, Brogaarden, Denmark), and tap water (lean chow, n=10-12). After 26 weeks, liver biopsies were performed for histological assessment of individual fibrosis and steatosis staging at baseline.

Mice were pretreated with enrofloxazin (Bayer, Germany) (5 mg/mL/1 mL/kg) one day before being biopsied. Prior to biopsy, mice were anesthetized with isoflurane (2%-3%) in 100% oxygen. A small abdominal incision in the midline was made and the left lateral lobe of the liver was exposed. A cone shaped wedge of liver tissue (50-100 mg) was excised from the distal portion of the lobe fixed in 4% paraformaldehyde for histology. The biopsy procedure previously described by Clapper et al. was refined using electrocoagulation of the cut surface of the liver by means of bipolar coagulation using ERBE VIO 1000 electrosurgical unit (ERBE, United States). The liver was returned to the abdominal cavity, abdominal wall was sutured and skin stapled. Carprofen (Pfizer, United States) (5 mg/mL-0.01 mL/10 g) and enrofloxazin (5 mg/mL-1 mL/kg) were administered intraperitoneal at the time of surgery and at post-operation day one and two, to control postoperative pain relief and infection, respectively. Following biopsy procedure animals were single housed and kept on AMLN diet for 3 weeks to recover. Stratification and randomization into study groups of 10-12 animals was based on individual disease staging as assessed by baseline liver biopsies.

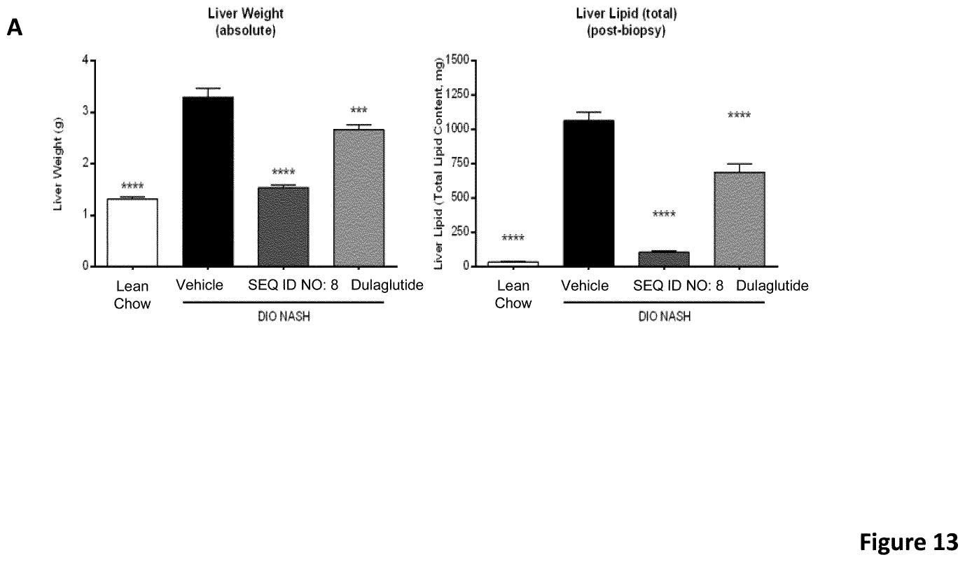

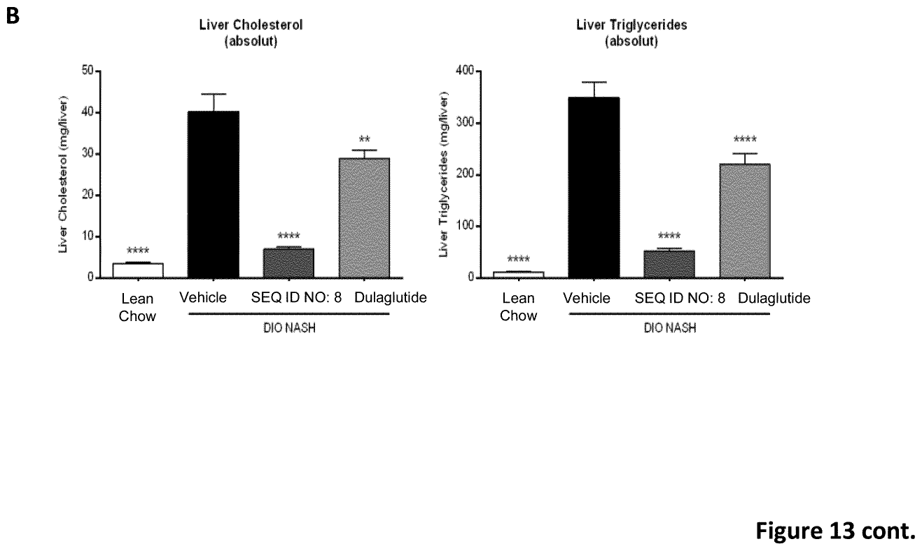

Animals were then treated with 50 mg/kg GLP-1RA/FGF21 Fc fusion protein, 0.6 mg/kg dulaglutide, or vehicle (PBS) once-weekly subcutaneously injections for further 8 weeks either on AMLN or chow diet. Subsequently animals were euthanized, liver weights were determined, and liver tissue collected for histological and biochemical analyses (see FIG. 13).

Histology Assessment and Digital Image Analysis

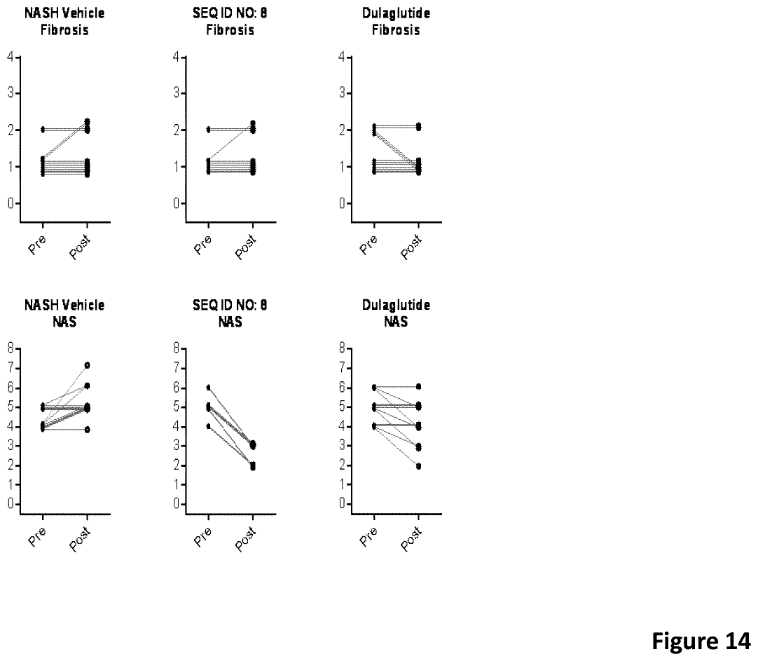

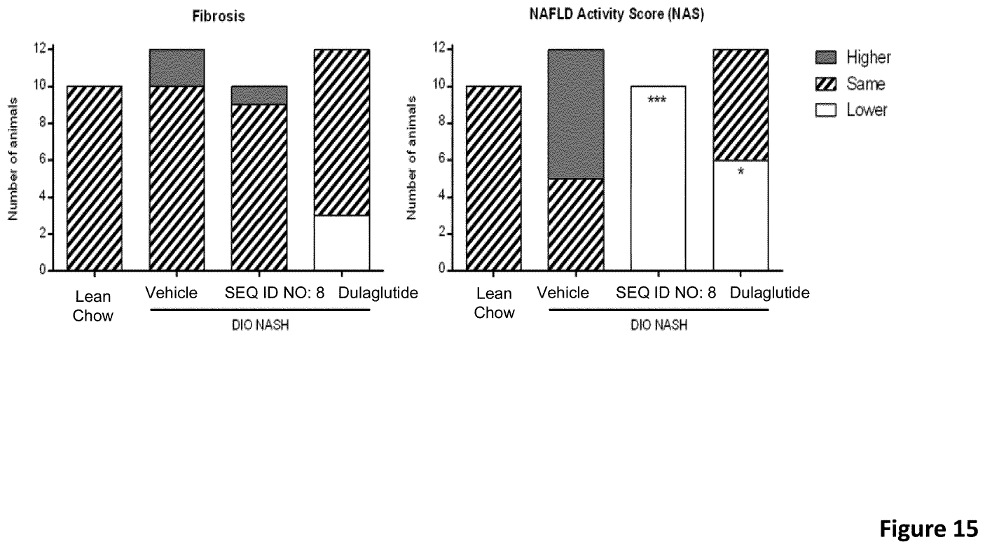

Baseline liver biopsy and terminal samples were collected from the left lateral lobe (about 100 mg) and fixed overnight in 4% paraformaldehyde. Liver tissue was paraffin embedded and sectioned (3 μm thickness). To assess hepatic morphology and fibrosis, sections were stained with Hematoxylin and Eosin and Sirius Red, respectively, followed by analysis with Visiomorph software (Visiopharm, Denmark). Histological assessment and scoring was performed by a pathologist blinded to the study. NAFLD activity score (NAS) (steatosis, inflammation, ballooning degeneration) and fibrosis stage were performed using the clinical criteria outlined by Kleiner et al. (2005) Hepatology 41: 1313-1321. Data are presented in two different formats in FIG. 14 and FIG. 15.

The fusion protein of SEQ ID NO: 8 clearly shows effects on liver weight, liver total lipid content, liver cholesterol and triglyceride content and NAFLD activity score which are superior to those of GLP-1 agonism alone, the latter being exemplified by the effect of dulaglutide.

1. A GLP-1R (Glucagon-Like Peptide-1 Receptor) agonistic peptide having a GLP-1R agonistic activity which is about 9- to about 531-fold reduced as compared to GLP-1R agonistic activity of a native GLP-1(7-36) (SEQ ID NO: 260), wherein the GLP-1R agonistic peptide comprises or consists of the amino acid sequence
(SEQ ID NO: 635)
X₁-G-E-G-T-F-T-S-D-X₁₀-S-X₁₂-X₁₃-L-X₁5-X₁₆-X₁₇-X₁₈-X₁₉-
X₂₀-X₂₁-F-X₂₃-E-W-L-X₂₇-X₂₈-X₂₉-G,
wherein
X₁ is H, Y or F,
X₁₀ is K or L,
X₁₂ is K, I or Q,
X₁₃ is Q or L,
X₁₅ is E, A or D,
X₁₆ is E, K or S,
X₁₇ is E, R or Q,
X₁₈ is L, A or R,
X₁₉ is V, A or F,
X₂₀ is R, H, Q, K or I,
X₂₁ is L, E, H or R,
X₂₃ is I, Y or F,
X₂₇ is I, L, K or E,
X₂₈ is A, K, N or E, and
X₂₉ is G, T, K or V;
wherein, optionally, the amino acid sequence further comprises at least one additional amino acid residue at its N-terminus; and
wherein, optionally, the amino acid sequence further comprises a peptide extension consisting of up to about 12, about 11 or about 10 amino acid residues at its C-terminus.

2. The GLP-1R agonistic peptide according to claim 1, comprising or consisting of the amino acid sequence
(SEQ ID NO: 636)
H-G-E-G-T-F-T-S-D-X₁₀-S-K-Q-L-E-E-E-X₁₈-V-X₂₀-L-F-I-
E-W-L-K-A-X₂₉-G,
wherein
X₁₀ is K or L,
X₁₈ is A or R,
X₂₀ is R or Q, and
X₂₉ is G or T;
wherein, optionally, the amino acid sequence further comprises at least one additional amino acid residue at its N-terminus; and
wherein, optionally, the amino acid sequence further comprises a peptide extension consisting of up to about 12, about 11 or about 10 amino acid residues at its C-terminus.

3. The GLP-1R agonistic peptide according to claim 1, wherein the at least one additional amino acid residue is G or A.

4. The GLP-1R agonistic peptide according to claim 1, wherein the peptide extension consists of an amino acid sequence selected from the group consisting of SEQ ID NOs: 566 to 621.

5. The GLP-1R agonistic peptide according to claim 1, wherein the GLP-1R agonistic peptide has a GLP-1R agonistic activity which is about 9- to about 531-fold reduced as compared to the GLP-1R agonistic activity of the native GLP-1(7-36) (SEQ ID NO: 260) when the GLP-1R agonistic peptide is in its isolated form and/or when the GLP-1R agonistic peptide is part of a fusion molecule.

6. The GLP-1R agonistic peptide according to claim 1, wherein the GLP-1R agonistic peptide has a GLP-1R agonistic activity which is about 10- to about 500-fold or about 15- to about 500-fold or about 20- to about 500-fold or about 50- to about 500-fold or about 100- to about 500-fold or about 100- to about 300-fold reduced as compared to the GLP-1R agonistic activity of the native GLP-1(7-36) (SEQ ID NO: 260).

7. A GLP-1R agonistic peptide comprising or consisting of an amino acid sequence selected from the group consisting of SEQ ID NOs: 261 to 552 and 554 to 565.

8. A GLP-1R agonistic peptide comprising or consisting of an amino acid sequence of SEQ ID NO: 261 or comprising or consisting of an amino acid sequence of SEQ ID NO: 262.

9. A combination comprising a GLP-1R agonistic peptide according to claim 1, and at least one other active pharmaceutical ingredient.

10. A fusion molecule comprising a GLP-1R agonistic peptide according to claim 1, and at least one other active pharmaceutical ingredient.

11. A nucleic acid molecule encoding a GLP-1R agonistic peptide according to claim 1.

12. A host cell containing a nucleic acid molecule according to claim 11.

13. A pharmaceutical composition comprising a GLP-1R agonistic peptide according to claim 1.

14. A kit comprising a GLP-1R agonistic peptide according to claim 1.

15. A GLP-1R agonistic peptide according to claim 1 for use as a medicament.

16. A GLP-1R agonistic peptide according to claim 1 for use in the treatment of a disease or disorder selected from the group consisting of obesity, being overweight, metabolic syndrome, diabetes mellitus, hyperglycemia, dyslipidemia, Non-Alcoholic SteatoHepatitis (NASH) and atherosclerosis.

17. The GLP-1R agonistic peptide, the combination, the fusion molecule, the nucleic acid molecule, the host cell or the pharmaceutical composition for use according to claim 16, wherein the diabetes mellitus is type 1 diabetes mellitus or type 2 diabetes mellitus.
